(19) United States
(12) Patent Application Publication
(10) Pub. No.: US 2018/0193409 A1
(43) Pub. Date: Jul. 12, 2018

(54) PEPTIDYL TPOR ANTAGONISTS AND USES THEREOF

(71) Applicant: POLYMERS CRC LIMITED, Notting Hill, Victoria (AU)

(72) Inventors: David HAYLOCK, Clayton, Victoria (AU); Anna TARASOVA, Clayton, Victoria (AU); David WINKLER, Clayton, Victoria (AU)

(73) Assignee: POLYMERS CRC LIMITED, Notting Hill, Victoria (AU)

(21) Appl. No.: 15/741,913

(22) PCT Filed: Jul. 6, 2016
(86) PCT No.: PCT/AU2016/050586

§ 371 Date: Jan. 4, 2018

(30) Foreign Application Priority Data
Jul. 6, 2015 (AU) 2015902665

(51) Int. Cl. A61K 38/12; A61K 38/10; A61K 38/03; A61P 7/00; A61P 35/02

(52) U.S. Cl. CPC A61K 38/12; A61K 38/10; A61P 35/02; A61P 7/00; A61K 38/03

(57) ABSTRACT

The present invention provides a method of treating a disease or condition associated with signalling via the TPO receptor. The method comprises the step of administering an effective amount of a peptidyl TPO receptor antagonist to a subject in need thereof, wherein the peptidyl TPOR receptor antagonist is a cyclic or linear peptidyl compound comprising the following structural formula (I): X_{bb}-Xₐₐ-X_{cc} wherein X_{bb} represents a residue of an amino acid selected from arginine (R) and lysine (K); Xₐₐ represents a residue of an amino acid selected from glutamine (Q), asparagine (N), aspartic acid (D), and glutamic acid (E); X_{cc} represents a residue of an amino acid selected from tryptophan (W), phenylalanine (F), tyrosine (Y), and histidine (H); or a salt thereof.

### FIELD OF THE INVENTION

The present invention relates generally to thromopoietin (TPO) mimetics, and more specifically TPO peptidyl mimetic compounds useful in the regulation of thrombopoiesis and megakaryocytopoiesis, and in the treatment of diseases or conditions associated with signalling via the TPO receptor (TPOR).

### BACKGROUND OF THE INVENTION

Megakaryocytes (bone marrow cells) are derived from hematopoietic stem and progenitor cells in the bone marrow. These pluripotent stem cells and committed progenitors live in the marrow sinusoids and are capable of producing all types of blood cells depending on the signals they receive. The primary signal for megakaryocyte production comes from TPO (thrombopoietin). Thus, TPO is necessary for inducing differentiation of progenitor cells in the bone marrow towards a final megakaryocyte phenotype.

TPO is a cytokine (a 353-amino acid protein, the gene of which is located on chromosome 3p27) whose main biological effects is as a major mediator of megakaryocyte (bone marrow cell) growth and platelet production. Platelets (thrombocytes) are an essential component in blood clotting and when a subject experiences low levels of platelets (thrombocytopenia) they risk death from severe haemorrhaging. TPO, and thus other molecules which mimic TPO (“TPO mimetics”), have potential utility in the diagnosis and treatment of a range of haematological disorders, including diseases like thrombocytopenia.

TPO is thought to act through binding to the cell surface (or extracellular receptor) c-Mpl or the TPO Receptor (TPOR). TPOR is a member of the hematopoietic growth factor receptor superfamily.

The extracellular domains of this family are typically composed of multiple β-sandwich molecules related to the fibroectin type III-immunoglobulin fold, characterised by a ligand domain formed from two adjacent β-sandwich structures.

The mechanism by which TPO activates TPOR is believed to be similar to the action of other hematopoietic cytokines which bind and induce receptor homodimerisation.

In general, the interaction of a protein ligand with its receptor often takes place at a relatively large interface. However, as demonstrated in the x-ray crystal structure of human growth hormone bound to its receptor, only a few key residues at the interface actually provide most of the binding energy. This and the fact that the bulk of the remaining protein ligand serves only to orient the binding epitopes in the correct topology makes it possible (in theory) to find active ligands of much smaller size.

Antagonists of the TPO receptor (TPOR) would be desireable for use in the treatment of diseases and conditions associated with signalling via the TPOR.

### SUMMARY OF THE INVENTION

The present invention is based inter alia on the recognition that specifically conserved residues which are deduced to be beneficial for the binding at TPOR can be effectively utilised to prepare antagonists of TPOR and are useful in treating diseases and conditions associated with signalling via the TPO receptor.

It has been determined that the tripeptide motif RQW (and substitutable variations thereof) facilitates binding to the cell surface (or extra cellular receptor) c-Mpl. Without wishing to be bound by theory the mode of action of the antagonist activity herein disclosed is proposed to be mediated by c-Mpl binding and the prevention of dimerisation of c-Mpl which in turn prevents downstream signal mediated cell survival and proliferation.

The present invention is predicated on the discovery that certain linear and cyclic peptides (in the hands of the present inventors) acts as TPOR antagonist and not as TPOR agonists which, in contrast, would induce receptor homodimerisation, signalling and proliferation. It has been discovered that the tripeptide motif RQW (or substitutable variations thereof) facilitates binding to the cell surface (or extra cellular receptor) c-Mpl. Without wishing to be bound by theory it is postulated that the antagonist activity discussed herein is caused by c-Mpl binding and the prevention of dimerisation of c-Mpl which in turn prevents downstream signal mediated cell survival and proliferation.

In one aspect the invention provides a method of treating a disease or condition associated with signalling via the TPO receptor, said method including the step of administering an effective amount of a peptidyl TPO receptor antagonist to a subject in need thereof, wherein the peptidyl TPOR receptor antagonist is characterised by the tripeptide motif RQW (or substitutable variations thereof).

In a further aspect the invention provides the use of a peptidyl TPO receptor antagonist which is characterised by the tripeptide motif RQW (or substitutable variations thereof) in the manufacture of a medicament for treating a disease or condition associated with signalling via the TPO receptor.

In a further aspect the invention provides a pharmaceutical composition for use in treating a disease or condition associated with signalling via the TPO receptor, wherein the composition comprises a peptidyl TPO receptor antagonist which is characterised by the tripeptide motif RQW (or substitutable variations thereof) and at least one pharmaceutically acceptable carrier, diluent or adjuvant.

In still a further aspect the invention provides a method of antagonising a TPO receptor in a cell, said method including the step of contacting the cell with an amount of a peptidyl TPO receptor antagonist, wherein the peptidyl TPO receptor antagonist is characterised with the tripeptide motif RQW (or substitutable variations thereof).

In an embodiment the method of antagonising a TPO receptor in a cell is conducted in vivo or in vitro or ex vivo.

In a further aspect the invention provides a method of identifying a peptidyl TPO receptor antagonist, the method including the steps of:
(1) contacting a cell expressing TPO receptor with a candidate peptidyl TPO receptor antagonist;
(2) detecting the ability of the candidate antagonist to inhibit the growth and/or proliferation of the cell,
   wherein inhibition of cell growth and/or proliferation is indicative of the TPO receptor antagonistic activity of the candidate.

In still further aspects the invention provides novel peptidyl TPO receptor antagonists and pharmaceutical compositions comprising same.

### BRIEF DESCRIPTION OF FIGURES

FIGS. 1(a) and (b) are graphical representations showing normalised proliferation as a function of the concentration of (a) non-purified large cyclic peptide (LCP) of Example 1 in media supplemented with (●) 6, (♦) 10 or (▴) 30 ng/mL recombinant, human thrombopoietin (rhTPO); and (b) purified large cyclic peptide (LCP-PM) of Example 1 in media supplemented with either 6 (●) or 10 (□) ng/mL of recombinant, human thrombopoietin (rhTPO).

FIG. 2 is a graphical representation of normalised proliferation as a function of the concentration of (●) non-purified large cyclic peptide (LCP) and (∘) purified large cyclic peptide (LCP-PM) of Example 1 in an agonist assay in the absence of rhTPO.

FIG. 3 is a graphical representation showing normalised proliferation as a function of the concentration of purified medium cyclic peptide (MCP-PM) of Example 2 in media supplemented with either 6 (●) or 10 (∘) ng/mL of recombinant, human thrombopoietin (rhTPO).

FIG. 4 is a graphical representation showing normalised proliferation as a function of the concentration of (●) non-purified medium cyclic peptide (MCP) and (∘) purified medium cyclic peptide (MCP-PM) of Example 2 in an agonist assay (i.e. in the absence of rhTPO).

FIG. 5 is a graphical representation showing as a function of the concentration of LCP, a constant concentration of TPO, the changing composition of cell types at day 14 of culture.

FIG. 6 is a graphical representation showing as a function of the concentration of LCP, a constant concentration of TPO, the changing composition of cell types at day 14 of culture.

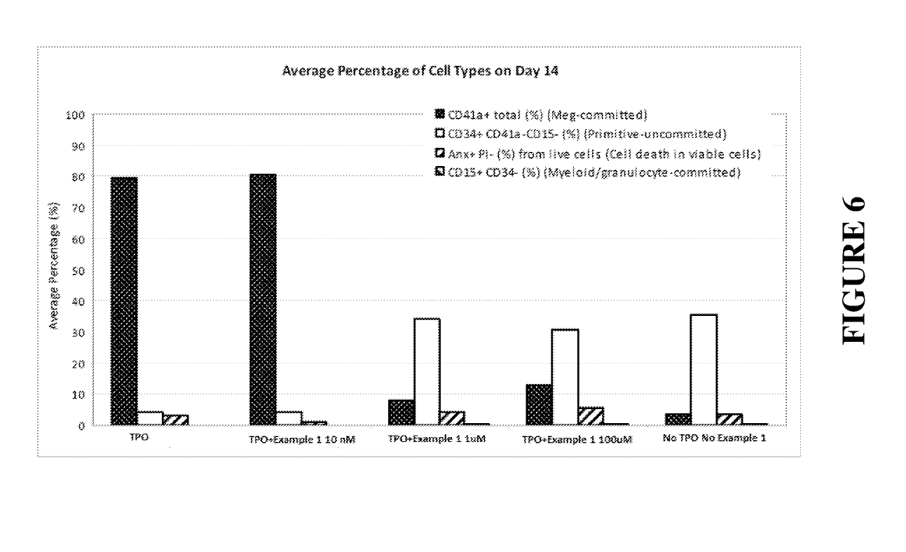

FIG. 7 is a graphical representation showing as a function of the concentration of LCP, a constant concentration of TPO, the changing composition of cell types at day 7 of culture.

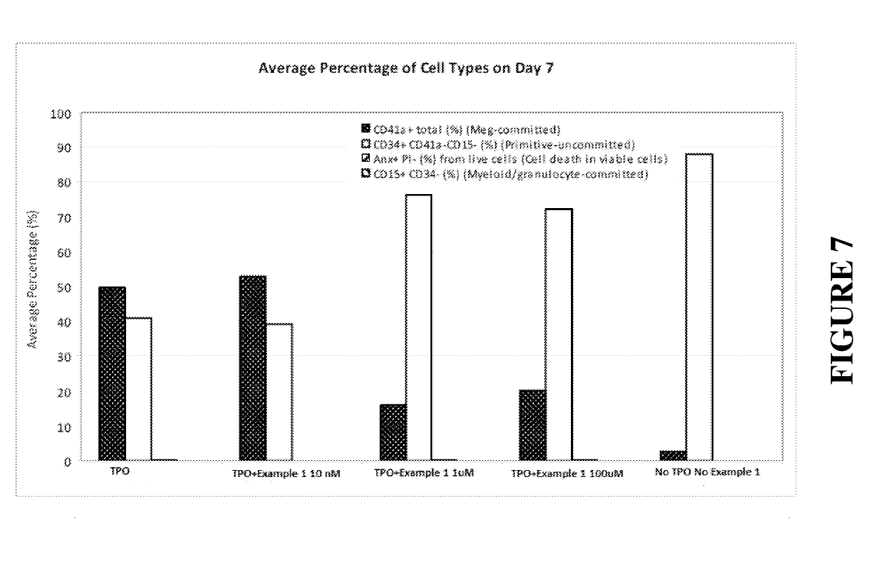

FIG. 8 is a graphical representation showing as a function of the concentration of LCP, a constant concentration of TPO, the changing composition of cell types at day 7 of culture.

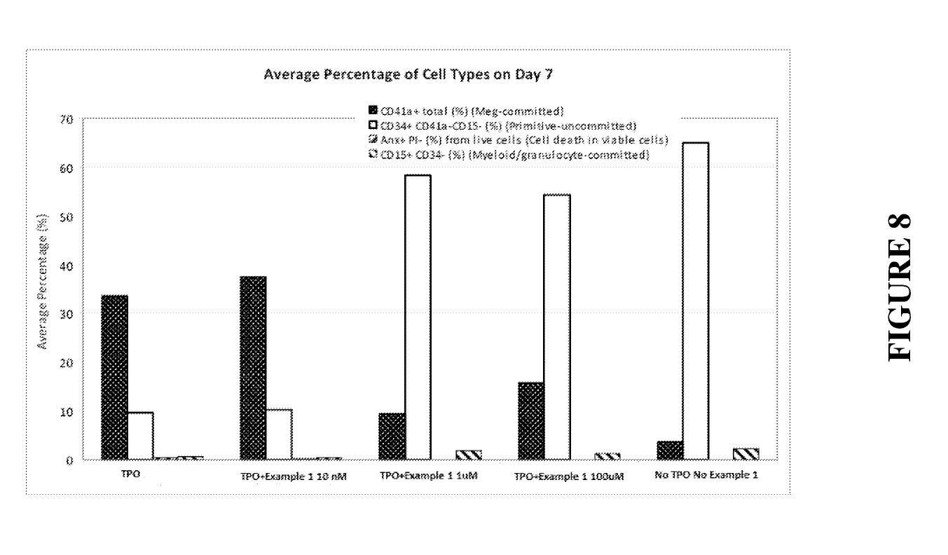

FIG. 9 is a graphical representation showing as a function of the concentration of LCP, a constant concentration of TPO, the changing composition of cell types at day 7 of culture.

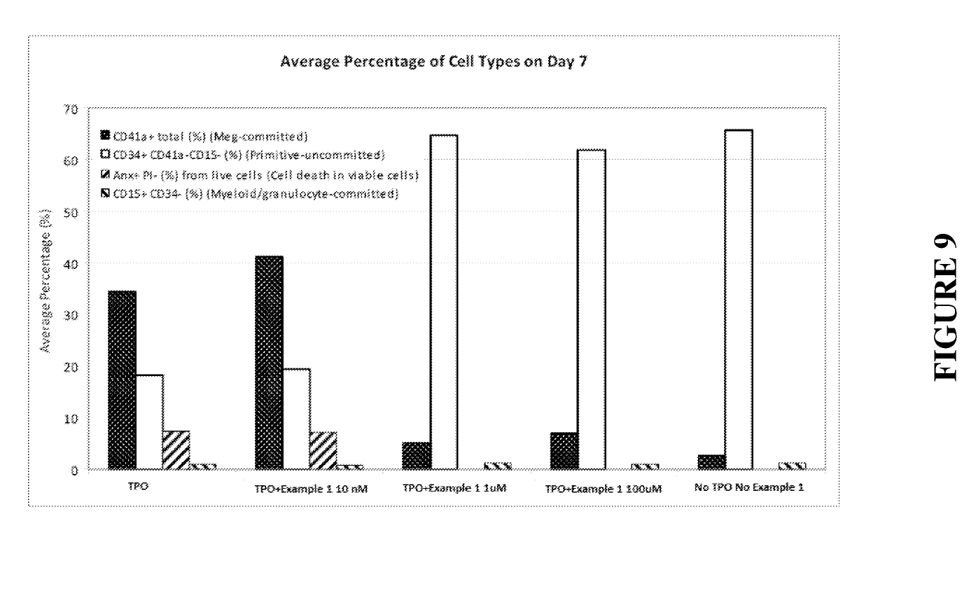

FIG. 10 is a graphical representation shown as a function of the concentration of LCP, and a constant concentration of TPO absolute numbers of each cell type at day 14 of culture.

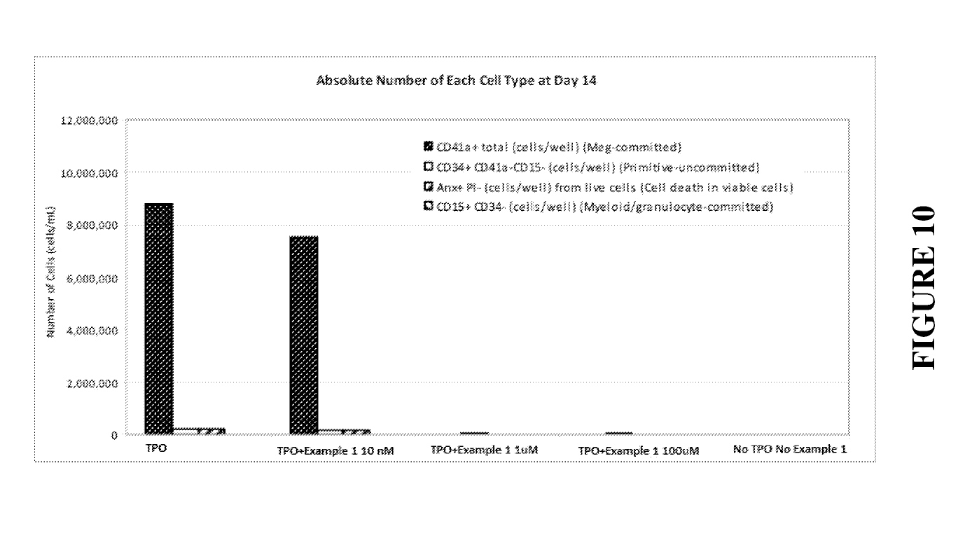

FIG. 11 is a graphical representation shown as a function of the concentration of LCP, and a constant concentration of TPO absolute numbers of each cell type at day 14 of culture.

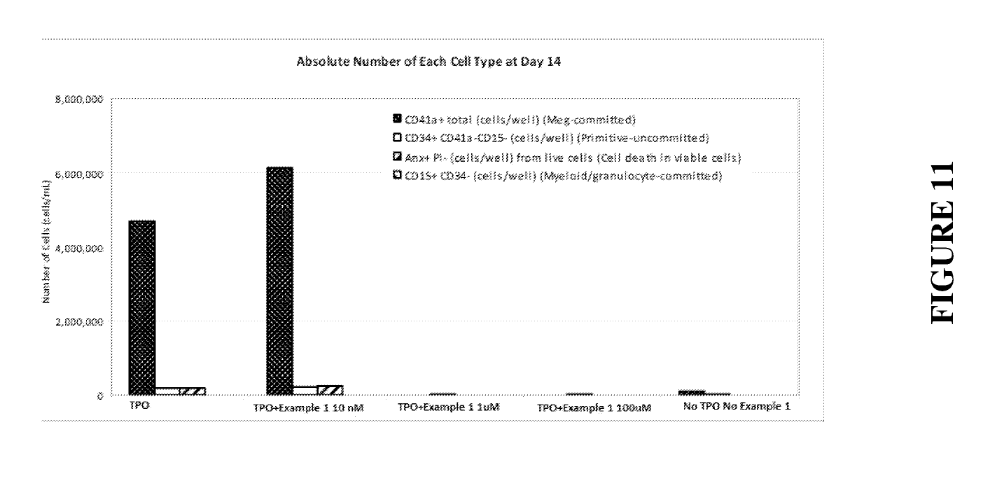

FIG. 12 is a graphical representation shown as a function of the concentration of LCP, and a constant concentration of TPO absolute numbers of each cell type at day 14 of culture.

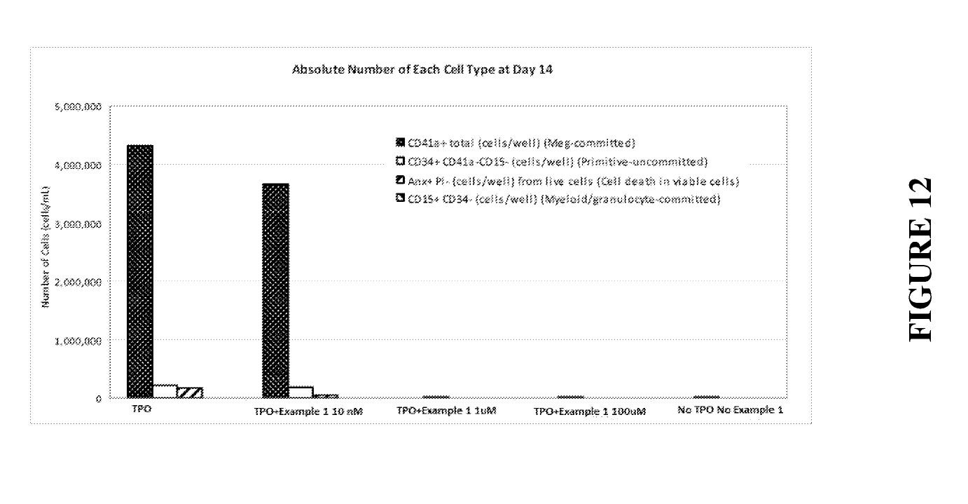

FIG. 13 is a graphical representation shown as a function of the concentration of LCP, and a constant concentration of TPO absolute numbers of each cell type at day 7 of culture.

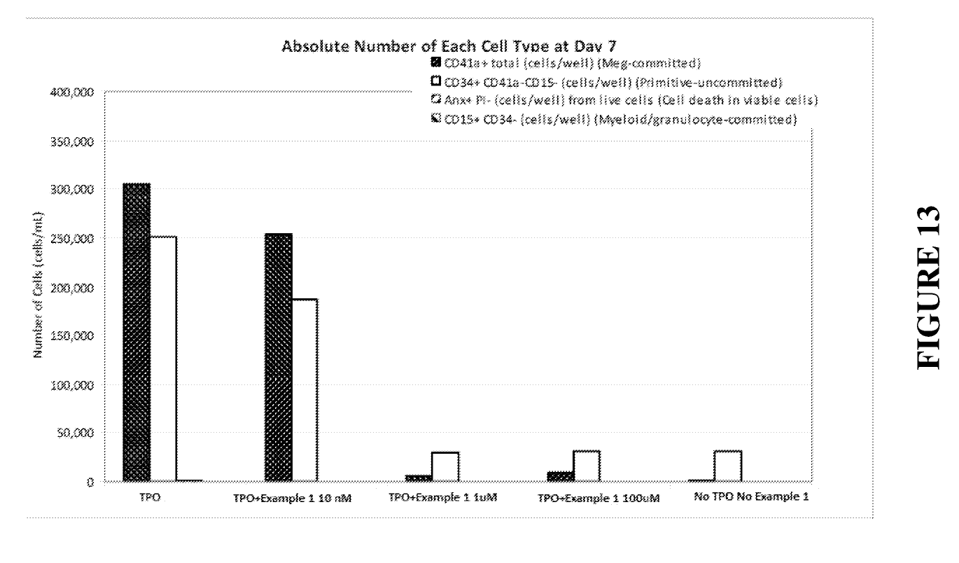

FIG. 14 is a graphical representation shown as a function of the concentration of LCP, and a constant concentration of TPO absolute numbers of each cell type at day 7 of culture.

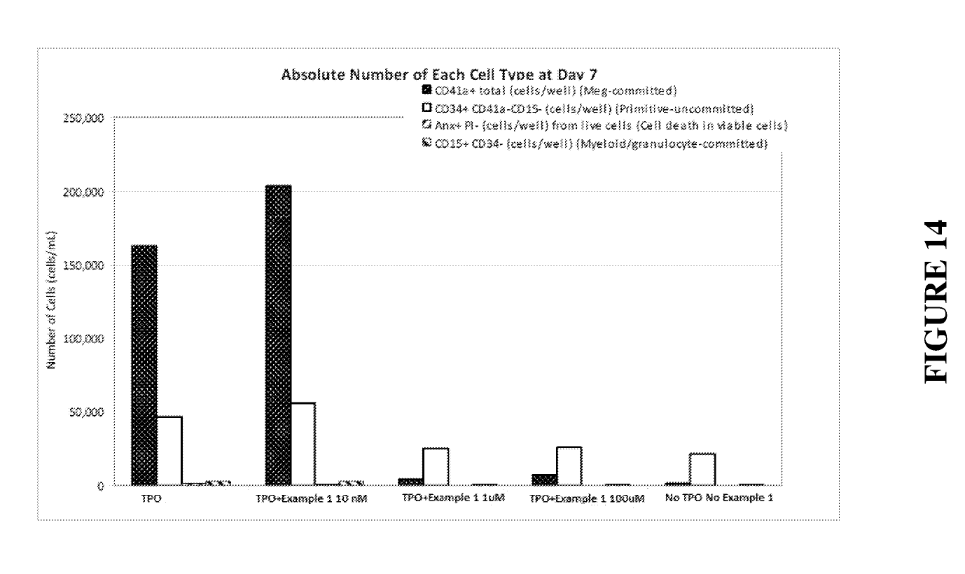

FIG. 15 is a graphical representation shown as a function of the concentration of LCP, and a constant concentration of TPO absolute numbers of each cell type at day 7 of culture.

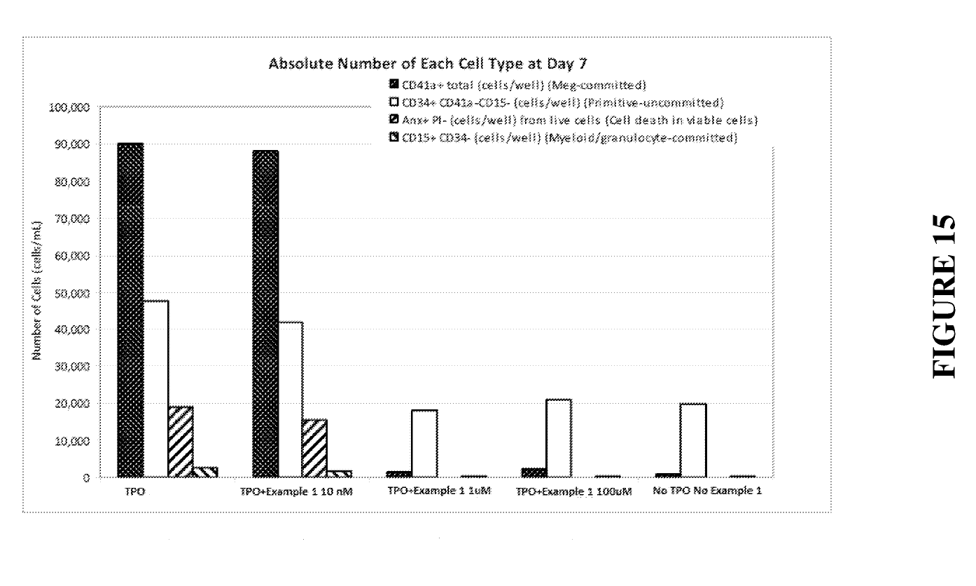

FIG. 16 is a graphical representation shown as a function of the concentration of TPO as a function of Example 1, the level of antagonist of the TPO receptor in the FD-Mpl cell.

FIG. 17 is a graphical representation shown as a function of the concentration of TPO as a function of LCP4 peptides, the level of antagonist of the TPO receptor in the FD-Mpl cell.

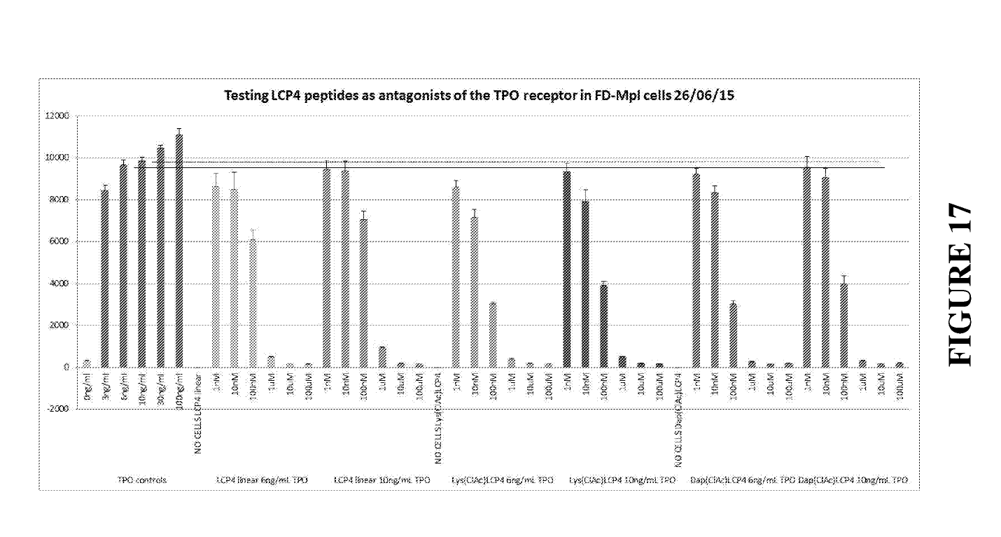

### DETAILED DESCRIPTION

The present inventors have found that particular peptidyl TPO mimetics which possess a RQW tripeptide motif (and substitutable variations thereof), act as suitable antagonists of the TPO receptor (or TPOR). In an embodiment, the TPO antagonists of the present invention possess a single RQW (or substitutable variations thereof) tripeptide motif.

The terms “TPO receptor” and “TPOR” are used interchangeably in this specification.

In an embodiment, the TPOR antagonist is selected from a cyclic or linear peptidyl compound comprising the following structural formula (I):

X_{bb}-Xₐₐ-X_{cc} formula (I)
wherein X_{bb} represents a residue of an amino acid selected from arginine (R) and lysine (K);
   Xₐₐ represents a residue of an amino acid selected from glutamine (Q), asparagine (N), aspartic acid (D), and glutamic acid (E);
   X_{cc} represents a residue of an amino acid selected from tryptophan (W), phenylalanine (F), tyrosine (Y), and histidine (H);
   or a salt thereof.

Specific variations of combinations contemplated are listed in Table 1 below:

TABLE 1

| X_{bb}-Xₐₐ-X_{cc} | X_{bb}-Xₐₐ-X_{cc} |
|---|---|
| R-Q-W | K-Q-W |
| R-Q-F | K-Q-F |
| R-Q-Y | K-Q-Y |
| R-Q-H | K-Q-H |
| R-E-W | K-E-W |
| R-E-F | K-E-F |
| R-E-Y | K-E-Y |
| R-E-H | K-E-H |
| R-N-W | K-N-W |
| R-N-F | K-N-F |
| R-N-Y | K-N-Y |
| R-N-H | K-N-H |
| R-D-W | K-D-W |
| R-D-F | K-D-F |
| R-D-Y | K-D-Y |
| R-D-H | K-D-H |

In an embodiment, the peptidyl TPOR antagonist is selected from a cyclic or linear peptidyl compound comprising the structural formula (Ia):

L-X_{bb}-Xₐₐ-X_{cc}-L formula (Ia)
wherein X_{bb} represents a residue of an amino acid selected from arginine (R) and lysine (K);
   Xₐₐ represents a residue of an amino acid selected from glutamine (Q), asparagine (N), aspartic acid (D), and glutamic acid (E);
   X_{cc} represents a residue of an amino acid selected from tryptophan (W), phenylalanine (F), tyrosine (Y), and histidine (H);
   or a salt thereof.

In an embodiment, the peptidyl TPOR antagonist is selected from a cyclic or linear peptidyl compound comprising the structural formula (Ib):

IEGPTL-X_{bb}-Xₐₐ-X_{cc}-L formula (Ib)
wherein X_{bb} represents a residue of an amino acid selected from arginine (R) and lysine (K);
   Xₐₐ represents a residue of an amino acid selected from glutamine (Q), asparagine (N), aspartic acid (D), and glutamic acid (E);
   X_{cc} represents a residue of an amino acid selected from tryptophan (W), phenylalanine (F), tyrosine (Y), and histidine (H);
   or a salt thereof.

In an embodiment, the peptidyl TPOR antagonist is selected from a cyclic or linear peptidyl compound comprising the structural formula (Ic):

PTL-X_{bb}-Xₐₐ-X_{cc}-LAARA formula (Ic)
wherein X_{bb} represents a residue of an amino acid selected from arginine (R) and lysine (K);
   Xₐₐ represents a residue of an amino acid selected from glutamine (Q), asparagine (N), aspartic acid (D), and glutamic acid (E);
   X_{cc} represents a residue of an amino acid selected from tryptophan (W), phenylalanine (F), tyrosine (Y), and histidine (H);
   or a salt thereof.

In an embodiment, the linear peptidyl TPOR antagonist is selected from a cyclic or linear peptidyl compound comprising or consisting of structural formula (Id):

IEGPTL-X_{bb}-Xₐₐ-X_{cc}-LAARA formula (Id)
wherein X_{bb} represents a residue of an amino acid selected from arginine (R) and lysine (K);
   Xₐₐ represents a residue of an amino acid selected from glutamine (Q), asparagine (N), aspartic acid (D), and glutamic acid (E);
   X_{cc} represents a residue of an amino acid selected from tryptophan (W), phenylalanine (F), tyrosine (Y), and histidine (H);
   or a salt thereof.

With respect to the peptidyl compounds of formulae (Ia)-(Id), in an embodiment, Xbb is arginine (R).

With respect to the peptidyl compounds of formulae (Ia)-(Id), in an embodiment, Xaa is glutamine (Q).

With respect to the peptidyl compounds of formulae (Ia)-(Id), in an embodiment, X_{cc} is tryptophan (W).

In an embodiment, the peptidyl TPOR antagonist of formula (I), (Ia), (Ib) (Ic) or (Id) is cyclic.

In an embodiment, the peptidyl TPOR antagonist is selected from a peptidyl compound of formula (I), (Ia), (Ib), (Ic) or (Id), comprising from 4 to 30 amino acid residues in length, preferably from 6 to 20 amino acid residues, for instance from 8 to 20, 10 to 20 or 15 to 20 amino acid residues.

In an embodiment, the TPOR antagonist of the present invention is selected from a cyclic peptidyl compound comprising the formula (II):

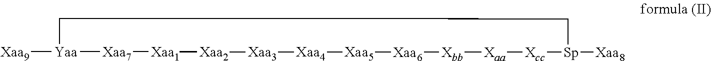

wherein Xaa₁, Xaa₂, Xaa₃, Xaa₄, Xaa₅, Xaa₆, Xaa₇, Xaa₈, and Xaa₉ are each independently a residue of any naturally or non-naturally occurring amino acid or are absent;
   X_{bb} represents a residue of an amino acid selected from arginine (R) and lysine (K);
   Xₐₐ represents a residue of an amino acid selected from glutamine (Q), asparagine (N), aspartic acid (D) and glutamic acid (E);
   X_{cc} represents a residue of an amino acid selected from tryptophan (W), phenylalanine (F), tyrosine (Y), and histidine (H);
   Yaa represents a residue of a natural or non-naturally occuring amino acid which is linked to Sp;
   Sp represents an amino acid spacer of 3-30 residues in length selected from naturally and non-naturally occurring amino acids which is linked to Yaa;
   or a salt or protected form thereof.

In an embodiment, the TPOR antagonist of the present invention is selected from a cyclic peptidyl compound comprising the formula (II′):

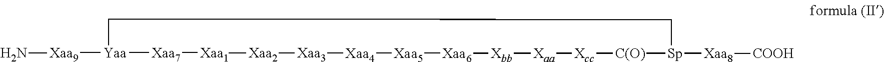

In an embodiment, the compound of formula (II) may be a cyclic peptidyl compound of formula (IIa):

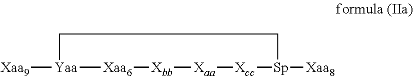

wherein:
Xaa6 is any naturally or non-naturally occurring amino acid; Xbb represents a residue of an amino acid selected from arginine (R) and lysine (K); Xaa represents a residue of an amino acid selected from glutamine (Q), asparagine (N), aspartic acid (D), glycine (G) and glutamic acid (E); Xcc represents a residue of an amino acid selected from tryptophan (W), phenylalanine (F), tyrosine (Y), and histidine (H); or a salt thereof; and Xaa9, Xaa8, Sp and Yaa are as defined above.

In an embodiment, in a compound of formula (IIa), Xaa6 is Gly, Ala, Val, Leu, Ile, Met, Pro or Phe; in an embodiment, Ala, Leu, Val or Ile; and in another embodiment, Leu.

A person skilled in the art would appreciate that in relation to compounds of formula (IIa) the various combinations of amino acids Xaa6, and Xbb represents a residue of an amino acid selected from arginine (R) and lysine (K); Xaa represents a residue of an amino acid selected from glutamine (Q), asparagine (N), aspartic acid (D), and glutamic acid (E); Xcc represents a residue of an amino acid selected from tryptophan (W), phenylalanine (F), tyrosine (Y), and histidine (H); or a salt thereof, as described above are within the scope and spirit of the present invention.

In an embodiment, the compound of formula (IIa) may be a cyclic peptidyl compound of formula (IIa″):

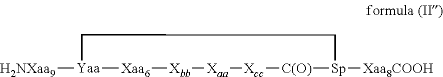

In an embodiment, the compound of formula (II) may be a cyclic peptidyl compound of formula (IIb):

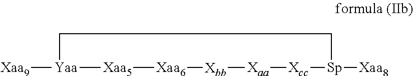

wherein: Xaa₆ and Xaa₅ are each independently any naturally or non-naturally occurring amino acid; X_{bb} represents a residue of an amino acid selected from arginine (R) and lysine (K); Xₐₐ represents a residue of an amino acid selected from glutamine (Q), asparagine (N), aspartic acid (D), and glutamic acid (E); X_{cc} represents a residue of an amino acid selected from tryptophan (W), phenylalanine (F), tyrosine (Y), and histidine (H); or a salt thereof; and Xaa₈, Xaa₉, Sp and Yaa are as defined above.

In another embodiment, in compounds of formula (IIb) Xaa5 is Ser, Thr, Asn, Gln, Tyr, Cys, Asp, or Glu; in an embodiment, Ser, Thr, Tyr, or Cys; and in another embodiment, Thr; Xaa6 is Gly, Aln, Val, Leu, Ile, Met, Pro, or Phe; in an embodiment, Ala, Leu, Val, or Ile; and in another embodiment, Leu.

A person skilled in the art would appreciate that in relation to compounds of formula (IIb) the various combinations of amino acids Xaa5, Xaa6, Xbb, Xaa, and Xcc as described above are within the scope and spirit of the present invention.

In an embodiment, the compound of formula (II) may be a cyclic peptidyl compound of formula (IIb″):

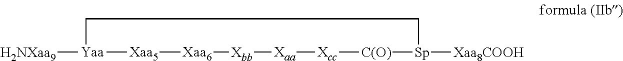

In another embodiment, the compound of formula (II) may be a cyclic peptidyl compound of formula (IIc):

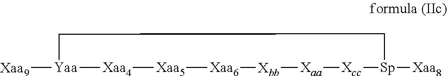

wherein:
Xaa4, Xaa5, and Xaa6 are each independently any naturally or non-naturally occurring amino acid; Xbb represents a residue of an amino acid selected from arginine (R) and lysine (K); Xaa represents a residue of an amino acid selected from glutamine (Q), asparagine (N), aspartic acid (D), and glutamic acid (E); X_{cc} represents a residue of an amino acid selected from tryptophan (W), phenylalanine (F), tyrosine (Y), and histidine (H); or a salt thereof; and Xaa₈, Xaa₉, Sp and Yaa are as defined above.
Xaa4 is Ala, Val, Leu, Ile, Pro or Phe; in an embodiment, Ala, Leu, Ile or Pro; and in another embodiment, Pro;
Xaa5 is Ser, Thr, Asn, Gln, Tyr, Cys, Asp or Glu; in an embodiment, Ser, Thr, Tyr or Cys; and in another embodiment, Thr; and
Xaa6 is Gly, Ala, Val, Leu, Ile, Met, Pro or Phe; in an embodiment, Ala, Leu, Val or Ile; and in another embodiment, Leu.

A person skilled in the art would appreciate that in relation to compounds of formula (IIc) the various combinations of amino acids Xaa₄, Xaa₅, Xaa₆, X_{bb}, Xₐₐ, and X_{cc} as described above are within the scope and spirit of the present invention.

In another embodiment, the compound of formula (IIc) may be a cyclic peptidyl compound of formula (IIc″):

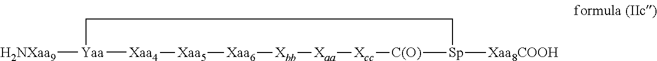

In another embodiment, in the compounds of formula (II), (IIa), (IIb), (IIc), and subformulae thereof:
Xaa₁ is Gly, Ala, Val, Leu, Ile, Met, Pro or Phe; in an embodiment, Ala, Leu, Val or Ile; and in another embodiment, Ile;
Xaa₂ is Ser, Thr, Asn, Gln, Tyr, Lys, Arg, His, Asp or Glu; in an embodiment, Gln, Asn, Asp or Glu; and in another embodiment, Glu;
Xaa₃ is Gly, Ala, Val, Leu, Ile, Pro or Phe; in an embodiment, Gly, Ala, Leu or Ile; and in another embodiment, Gly;
Xaa₄ is Ala, Val, Leu, Ile, Pro or Phe; in an embodiment, Ala, Leu, Ile or Pro; and in another embodiment, Pro;
Xaa₅ is Ser, Thr, Asn, Gln, Tyr, Cys, Asp or Glu; in an embodiment, Ser, Thr, Tyr or Cys; and in another embodiment, Thr;
Xaa₆ is Gly, Ala, Val, Leu, Ile, Met, Pro or Phe; in an embodiment, Ala, Leu, Val or Ile; and in another embodiment, Leu; and
Xaa₇ is preferably Gly.

A person skilled in the art would appreciate that in relation to compounds of formula (II), (IIa), (IIb) and (IIc), and subformulae thereof, the various combinations of amino acids Xaa₁, Xaa₂, Xaa₃, Xaa₄, Xaa₅, Xaa₆, X_{bb}, Xₐₐ, and X_{cc} as described above (as they occur) are within the scope and spirit of the present invention.

In an embodiment, in compounds of formula (II), (IIa), (IIb), and (IIc), and subformulae thereof, Xaa₈ and Xaa₉ are independently A or absent.

In an embodiment, in compounds of formula (II), (IIa), (IIb), and (IIc), and subformulae thereof, Xaa₈ is A and Xaa₉ is absent.

In an embodiment, in compounds of formula (II), (IIa), (IIb), and (IIc), and subformulae thereof, Xaa₈ is absent and Xaa₉ is A.

In an embodiment, in compounds of formula (II), (IIa), (IIb), and (IIc), and subformulae thereof, Xaa₈ and Xaa₉ are both absent.

In an embodiment, the compound of formula (II) may be or comprise a compound of formula (IIa′):

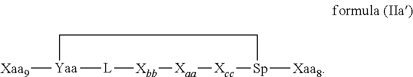

In an embodiment, the compound of formula (IIa′) may be or comprise a compound of formula (IIaa′):

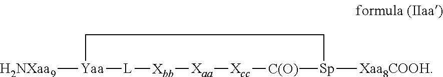

In an embodiment, the compound of formula (II) may be or comprise a compound of formula (IIb′):

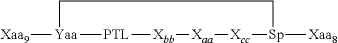

In an embodiment, the compound of formula (IIb′) may be or comprise a compound of formula (IIbb′):

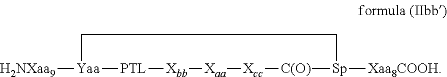

In an embodiment, the compound of formula (II) may be or comprise a compound of formula (IIc′):

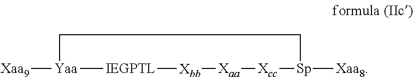

In an embodiment, the compound of formula (IIc′) may be or comprise a compound of formula (IIcc′):

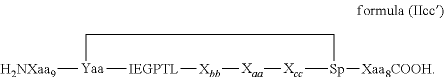

In an embodiment, the peptidyl TPOR antagonist of the present invention is selected from a cyclic peptidyl compound of formula (II), (Iia), (Iib), (Iic), (Iia′), (Iib′) and (Iic′) (and subformulae thereof), comprising from 10 to 40 amino acids residues in length, such as from 12-30 amino acid residues and including from 15-25 amino acid residues, such as 15, 16, 17, 18, 19, 20, 21, 22, 23, 24 or 25 amino acid residues.

In an embodiment, peptidyl TPOR antagonist of the present invention is selected from a cyclic peptidyl compound of formula (II), (Iia), (Iib), (Iic), (Iia′), (Iib′) and (Iic′) (and subformulae thereof), comprising 20 amino acid residues.

In another embodiment, peptidyl TPOR antagonist of the present invention is selected from a cyclic peptidyl compound of formula (II), (Iia), (Iib), (Iic), (Iia′), (Iib′) and (Iic′) (and subformulae thereof), comprising 19 amino acid residues.

In relation to compounds of formulae (II), (Iia), (Iib), (Iic), (Iia′), (Iib′) and (Iic′) (and subformulae thereof), Sp represents an amino acid spacer of 3 to 10 residues in length selected from naturally occurring or non-naturally occurring amino acids.

It is through the Sp group that the peptide of formula (II) is cyclised. The cyclised portion of the compound of formulae (II), (Iia), (Iib), (Iic), (Iia′), (Iib′) and (Iic′) (and subformulae thereof), may be cyclised through amide bonds along the peptide backbone of Sp.

In an embodiment, the TPOR antagonist is selected from a cyclic or linear peptidyl compound comprising the following structural formula (III):

RQW formula (III)

or a salt thereof.

In an embodiment, the peptidyl TPOR antagonist is selected from a cyclic or linear peptidyl compound comprising the structural formula (IIIa):

LRQWL formula (IIIa)

or a salt thereof.

In an embodiment, the peptidyl TPOR antagonist is selected from a cyclic or linear peptidyl compound comprising the structural formula (IIIb):

IEGPTLRQWL formula (IIIb)

or a salt thereof.

In an embodiment, the peptidyl TPOR antagonist is selected from a cyclic or linear peptidyl compound comprising the structural formula (IIIc):

PTLRQWLAARA formula (IIIc)

or a salt thereof.

In an embodiment, the linear peptidyl TPOR antagonist is selected from a cyclic or linear peptidyl compound comprising or consisting of structural formula (IIId):

IEGPTLRQLAARA formula (IIId)

or a salt thereof.

In an embodiment, the peptidyl TPOR antagonist of formula (III), (IIIa), (IIIb) or (IIIc) is cyclic.

In an embodiment, the peptidyl TPOR antagonist is selected from a peptidyl compound of formula (III), (IIIa), (IIIb) or (IIIc), comprising from 4 to 30 amino acid residues in length, preferably from 6 to 20 amino acid residues, for instance from 8 to 20, 10 to 20, or 15 to 20 amino acid residues.

In an embodiment, the TPOR antagonist of the present invention is selected from a cyclic peptidyl compound comprising the formula (IV):

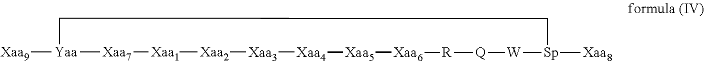

wherein Xaa₁, Xaa₂, Xaa₃, Xaa₄, Xaa₅, Xaa₆, Xaa₇, Xaa₈, and Xaa₉ are each independently a residue of any naturally or non-naturally occurring amino acid or are absent;
   Yaa represents a residue of a natural or non-naturally occurring amino acid which is linked to Sp;
   Sp represents an amino acid spacer of 3-30 residues in length selected from naturally and non-naturally occurring amino acids which is linked to Yaa;
   or a salt or protected form thereof.

In an embodiment, the TPOR antagonist of the present invention is selected from a cyclic peptidyl compound comprising the formula (IV″):

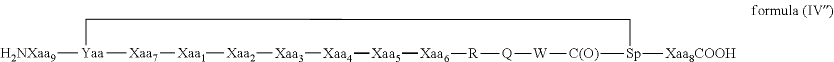

In an embodiment, the TPOR antagonist of the present invention is selected from a cyclic peptidyl compound comprising the formula (IV′):

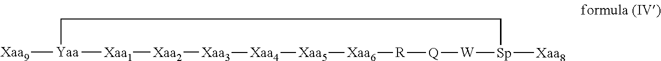

In an embodiment, the TPOR antagonist of the present invention is selected from a cyclic peptidyl compound comprising the formula (IV′″):

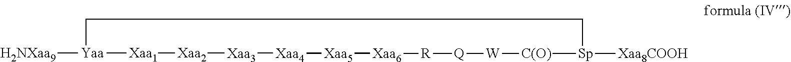

In an embodiment, the compound of formula (IV) may be a cyclic peptidyl compound of formula (IVa):

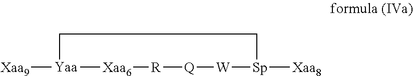

wherein:
Xaa₆ is any naturally or non-naturally occurring amino acid; and Xaa₇, Xaa₈, Xaa₉, Sp and Yaa are as defined above.

In an embodiment, in a compound of formula (IVa), Xaa₆ is Gly, Ala, Val, Leu, Ile, Met, Pro or Phe; in an embodiment, Ala, Leu, Val or Ile; and in another embodiment, Leu.

A person skilled in the art would appreciate that in relation to compounds of formula (IVa) the various combinations of amino acid Xaa₆ as described above are within the scope and spirit of the present invention.

In an embodiment, the compound of formula (IVa) may be a cyclic peptidyl compound of formula (IVa″):

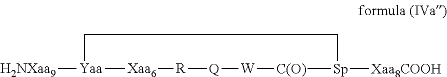

In an embodiment, the compound of formula (IV) may be a cyclic peptidyl compound of formula (IVb):

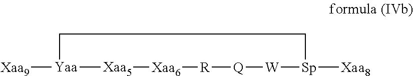

wherein: Xaa₆ and Xaa₅ are each independently any naturally or non-naturally occurring amino acid; and Xaa₈, Xaa₉, Sp and Yaa are as defined above.

In another embodiment, in compounds of formula (IVb) Xaa₅ is Ser, Thr, Asn, Gln, Tyr, Cys, Asp, or Glu; in an embodiment, Ser, Thr, Tyr, or Cys; and in another embodiment, Thr; Xₐₐ₆ is Gly, Aln, Val, Leu, Ile, Met, Pro, or Phe; in an embodiment, Ala, Leu, Val, or Ile; and in another embodiment, Leu.

A person skilled in the art would appreciate that in relation to compounds of formula (IVb) the various combinations of amino acids Xaa₅, and Xaa₆ as described above are within the scope and spirit of the present invention.

In an embodiment, the compound of formula (IVb) may be a cyclic peptidyl compound of formula (IVb″):

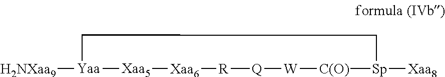

In another embodiment, the compound of formula (IV) may be a cyclic peptidyl compound of formula (IVc):

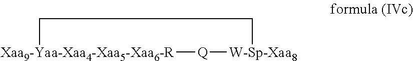

wherein Xaa₄, Xaa₅, and Xaa₆ are each independently any naturally or non-naturally occurring amino acid; and Xaa₈, Xaa₉, Sp and Yaa are as defined above.

In another embodiment, in the compounds of formula (IVc) Xaa₄ is Ala, Val, Leu, Ile, Pro or Phe; in an embodiment, Ala, Leu, Ile or Pro; and in another embodiment, Pro; Xaa₅ is Ser, Thr, Asn, Gln, Tyr, Cys, Asp or Glu; in an embodiment, Ser, Thr, Tyr or Cys; and in another embodiment, Thr; and Xaa₆ is Gly, Ala, Val, Leu, Ile, Met, Pro or Phe; in an embodiment, Ala, Leu, Val or Ile; and in another embodiment, Leu.

A person skilled in the art would appreciate that in relation to compounds of formula (IVc) the various combinations of amino acids Xaa₄, Xaa₅, Xaa₆ as described above are within the scope and spirit of the present invention.

In another embodiment, the compound of formula (IV) may be a cyclic peptidyl compound of formula (IVc″):

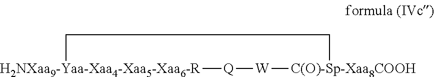

In another embodiment, in the compounds of formula (IV), (IVa), (IVb) and (IVc):
Xaa₁ is Gly, Ala, Val, Leu, Ile, Met, Pro or Phe; in an embodiment, Ala, Leu, Val or Ile; and in another embodiment, Ile;
Xaa₂ is Ser, Thr, Asn, Gln, Tyr, Lys, Arg, His, Asp or Glu; in an embodiment, Gln, Asn, Asp or Glu; and in another embodiment, Glu;
Xaa₃ is Gly, Ala, Val, Leu, Ile, Pro or Phe; in an embodiment, Gly, Ala, Leu or Ile; and in another embodiment, Gly;
Xaa₄ is Ala, Val, Leu, Ile, Pro or Phe; in an embodiment, Ala, Leu, Ile or Pro; and in another embodiment, Pro;
Xaa₅ is Ser, Thr, Asn, Gln, Tyr, Cys, Asp or Glu; in an embodiment, Ser, Thr, Tyr or Cys; and in another embodiment, Thr;
Xaa₆ is Gly, Ala, Val, Leu, Ile, Met, Pro or Phe; in an embodiment, Ala, Leu, Val or Ile; and in another embodiment, Leu; and
Xaa₇ is preferably Gly.

A person skilled in the art would appreciate that in relation to compounds of formula (IV), (IVa), (IVb) and (IVc) (and subformulae thereof) the various combinations of amino acids Xaa₁, Xaa₂, Xaa₃, Xaa₄, Xaa₅, Xaa₆, Xaa₇, Xaa₈, and Xaa₉ as described above (as they occur) are within the scope and spirit of the present invention.

In an embodiment, in compounds of formula (IV), (IVa), (IVb), and (IVc) (and subformulae thereof) Xaa₈ and Xaa₉ are independently A or absent.

In an embodiment, in compounds of formula (IV), (IVa), (IVb), and (IVc) (and subformulae thereof) Xaa₈ is A and Xaa₉ is absent.

In an embodiment, in compounds of formula (IV), (IVa), (IVb), and (IVc) (and subformulae thereof) Xaa₈ is absent and Xaa₉ is A.

In an embodiment, in compounds of formula (IV), (IVa), (IVb), and (IVc) (and subformulae thereof) Xaa₈ and Xaa₉ are both absent.

In an embodiment, the compound of formula (IV) may be or comprise a compound of formula (IVa′):

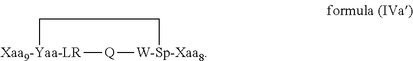

In an embodiment, the compound of formula (IVa′) may be or comprise a compound of formula (IVaa′):

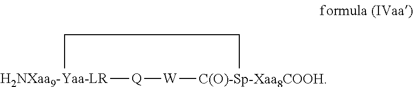

In an embodiment, the compound of formula (IV) may be or comprise a compound of formula (IVb′):

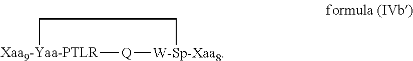

In an embodiment, the compound of formula (IVb′) may be or comprise a compound of formula (IVbb′):

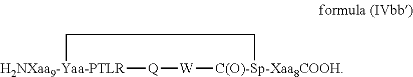

In an embodiment, the compound of formula (IV) may be or comprise a compound of formula (IVc′):

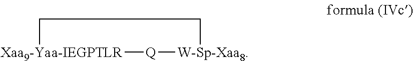

In an embodiment, the compound of formula (IVc′) may be or comprise a compound of formula (IVcc′):

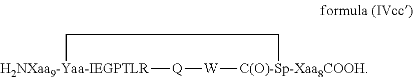

In an embodiment, the compound of formula (IV) may be or comprise a compound of formula (IVd′):

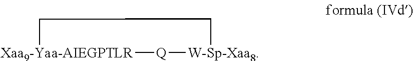

In an embodiment, the compound of formula (IVd′) may be or comprise a compound of formula (IVdd′):

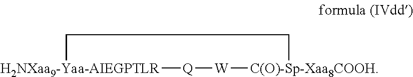

In an embodiment, the peptidyl TPOR antagonist of the present invention is selected from a cyclic peptidyl compound of formula (IV), (IVa), (IVb), (IVc), (IVa′), (IVb′) and (IVc′) (and subformulae thereof) comprising from 10 to 40 amino acids residues in length, such as from 12-30 amino acid residues and including from 15-25 amino acid residues, such as 15, 16, 17, 18, 19, 20, 21, 22, 23, 24 or 25 amino acid residues.

In an embodiment, peptidyl TPOR antagonist of the present invention is selected from a cyclic peptidyl compound of formula (IV), (IVa), (IVb), (IVc), (IVa′), (IVb′) and (IVc′) (and subformulae thereof) comprising 20 amino acid residues.

In another embodiment, peptidyl TPOR antagonist of the present invention is selected from a cyclic peptidyl compound of formula (IV), (IVa), (IVb), (IVc), (IVa′), (IVb′) and (IVc′) (and subformulae thereof) comprising 19 amino acid residues.

In relation to compounds of formulae (IV), (IVa), (IVb), (IVc), (IVa′), (IVb′) and (IVc′) (and subformulae thereof), Sp represents an amino acid spacer of 3 to 10 residues in length selected from naturally occurring or non-naturally occurring amino acids.

It is through the Sp group that the peptide of formula (IV) is cyclised. The cyclised portion of the compound of formulae (IV), (IVa), (IVb), (IVc), (IVa′), (IVb′) and (IVc′) may be cyclised through amide bonds along the peptide backbone of Sp, for instance to Yaa.

Sp may also include one or more residues which are connected through their side chains for instance to Yaa. For example, the residues may be connected by virtue of a disulfide bond created between two cysteine residues. In another example, the residues may be connected by an amide bond formed between the side chains of aspartic acid and lysine. Various ways of connecting two amino acid residues are known to a person skilled in the art.

In an embodiment, the TPOR antagonist is a cyclic peptide comprising at least three residues adjacent to W in RQW.

In an embodiment, the TPOR antagonist is a cyclic peptide comprising three residues adjacent to W in RQW.

In an embodiment, the TPOR antagonist is a cyclic peptide comprising four residues adjacent to W in RQW.

In an embodiment, the TPOR antagonist is a cyclic peptide comprising five residues adjacent to W in RQW.

In an embodiment, the TPOR antagonist is a cyclic peptide comprising six residues adjacent to W in RQW.

In an embodiment, the TPOR antagonist is a cyclic peptide comprising seven residues adjacent to W in RQW.

In an embodiment, the TPOR antagonist is a cyclic peptide comprising eight residues adjacent to W in RQW.

In an embodiment, the TPOR antagonist is a cyclic peptide comprising at least three residues adjacent to R in RQW.

In an embodiment, the TPOR antagonist is a cyclic peptide comprising three residues adjacent to R in RQW.

In an embodiment, the TPOR antagonist is a cyclic peptide comprising four residues adjacent to R in RQW.

In an embodiment, the TPOR antagonist is a cyclic peptide comprising five residues adjacent to R in RQW.

In an embodiment, the TPOR antagonist is a cyclic peptide comprising six residues adjacent to R in RQW.

In an embodiment, the TPOR antagonist is a cyclic peptide comprising seven residues adjacent to R in RQW.

In an embodiment, the TPOR antagonist is a cyclic peptide comprising eight residues adjacent to R in RQW.

In an embodiment, the TPOR antagonist is a cyclic peptide comprising at least three residues adjacent to R and at least three residues adjacent to W in RQW.

In an embodiment, the TPOR antagonist is a cyclic peptide comprising at least four residues adjacent to R and at least four residues adjacent to W in RQW.

In an embodiment, the TPOR antagonist is a cyclic peptide comprising at least five residues adjacent to R and at least five residues adjacent to W in RQW.

In an embodiment, the TPOR antagonist is a cyclic peptide comprising at least six residues adjacent to R and at least six residues adjacent to W in RQW.

In an embodiment, the TPOR antagonist is a cyclic peptide comprising at least seven residues adjacent to R and at least seven residues adjacent to W in RQW.

In an embodiment, the TPOR antagonist is a cyclic peptide comprising at least eight residues adjacent to R and at least eight residues adjacent to W in RQW. In an embodiment, it is desireable to form the cyclic peptides with more stronger and physiologically robust bonds than a disulphide bond. Other avenues for forming cyclic peptides would be well known to those in the art and may include the formation of thioethers, triazoles (by use of ‘click chemistry’), amide, alkynes (by metathesis reaction), alkenes, alkanes, and diselenides bonds (formed between two selenocysteine residues). Examples of cyclic peptides of the invention which utilize such bond forming chemistries are shown below:

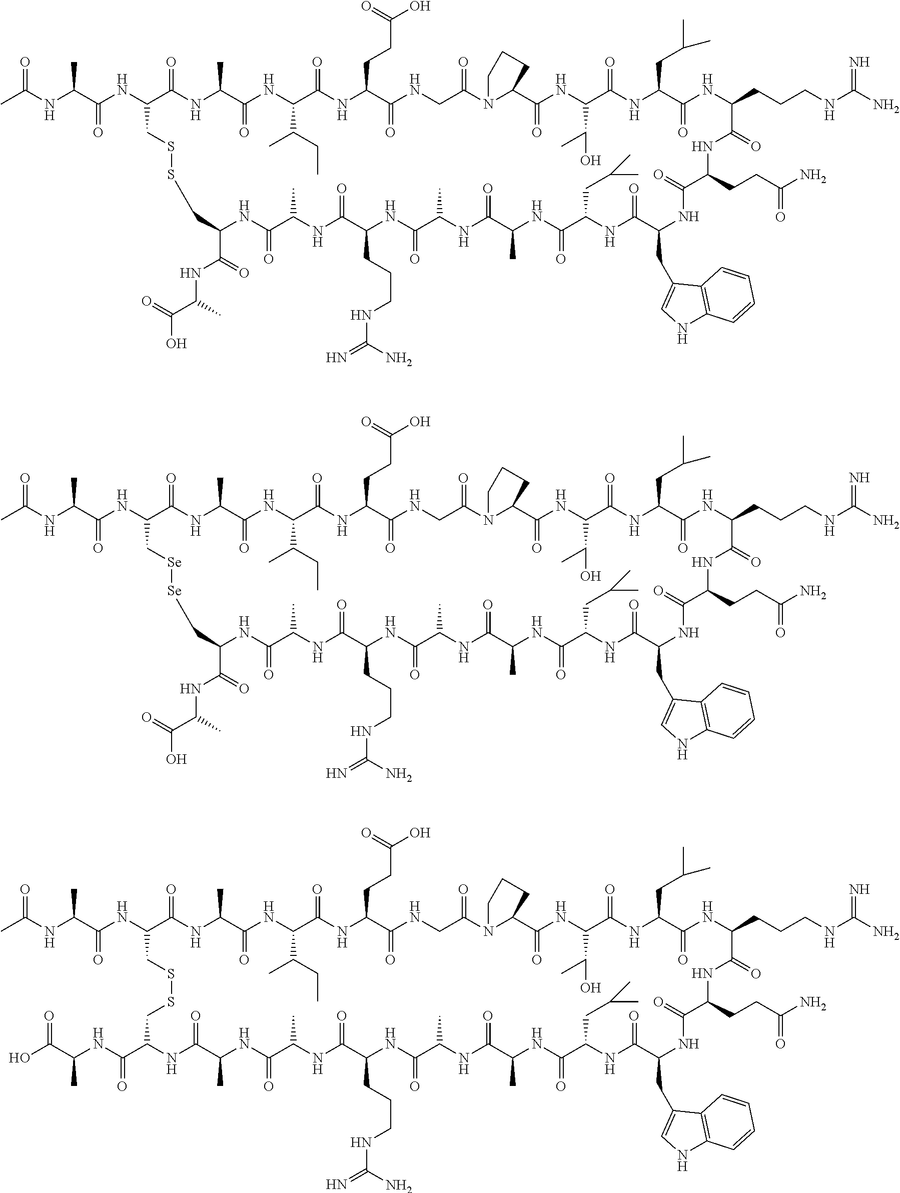

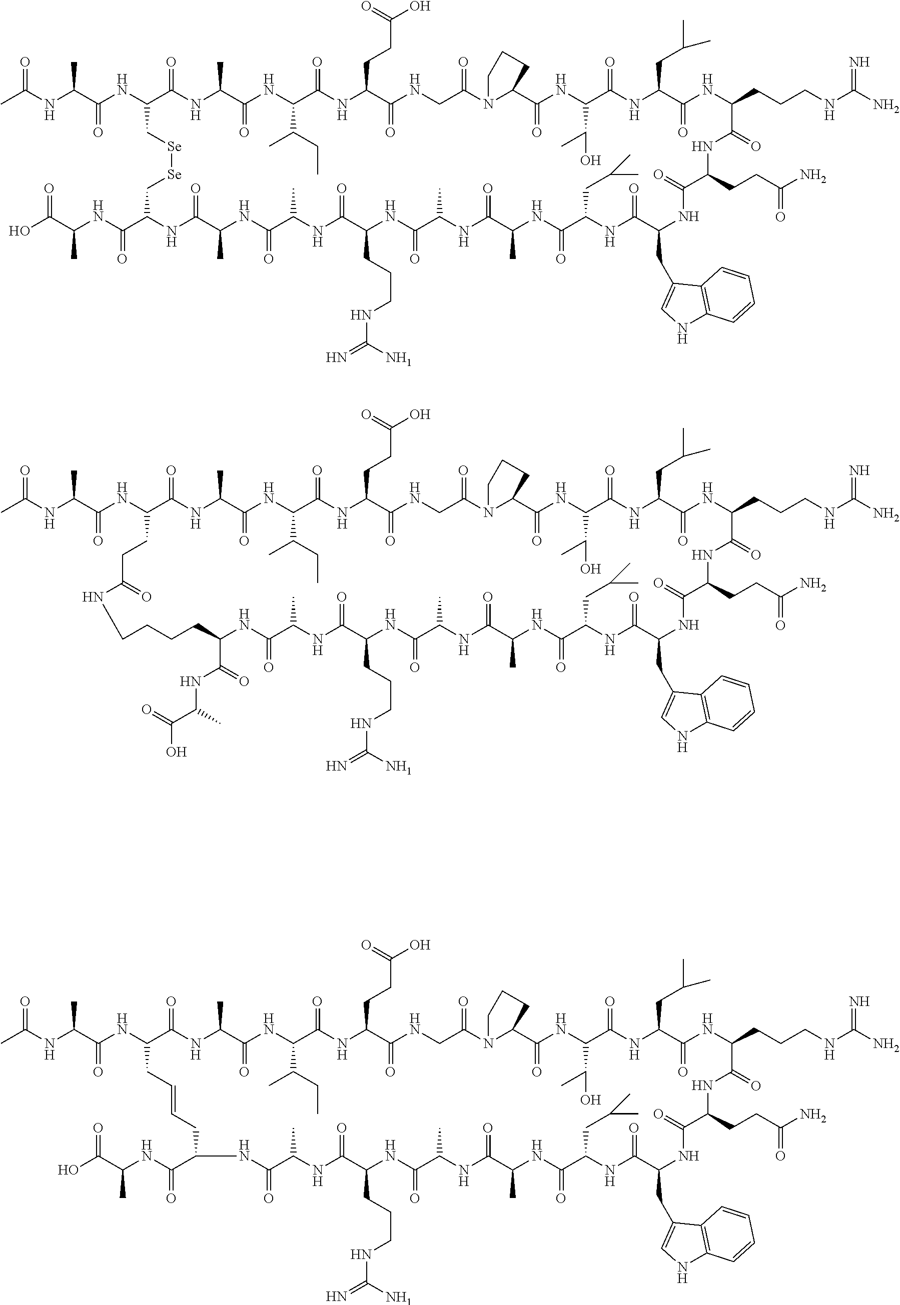

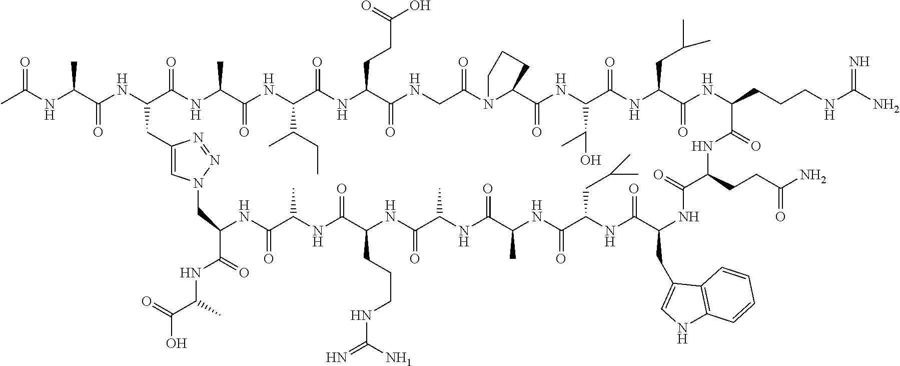

The Sp amino acids may be optionally substituted, for example through appending additional amino acids or other organic substituents. For example, one or more amino acids may be substituted at the nitrogen along the peptide backbone. In another example, one or more side chains of the amino acids may bear additional substituents.

In an embodiment, the amino acid in Sp adjacent to W in RQW is selected from Gly, Ala, Val, Leu, Ile, Met, Pro or Phe; in an embodiment, Ala, Leu, Val or Ile; and in an embodiment, Leu.

In another embodiment the Sp group comprises or consists of -L-.

In another embodiment the Sp group comprises or consists of -LA-.

In another embodiment the Sp group comprises or consists of -LAA-.

In another embodiment the Sp group comprises or consists of -LAAR-.

In another embodiment the Sp group comprises or consists of -LAARA-.

In another embodiment the Sp group comprises or consists of -LAARAA-.

In another embodiment the Sp group comprises or consists of -LAARAAC-.

In another embodiment the Sp group comprises or consists of -LAARAACA.

Considerable variation in the peptide sequence of Sp is possible. Since this spacing region may not necessarily bind to or occlude the primary active site of the peptide it can be modified to alter physiochemical properties, or otherwise improve the properties of the peptide, such as by improving stability.

In an embodiment Yaa is a residue of cysteine (C), or another amino acid residue (natural or non-natural) which has been suitably modified to enable linking to Sp.

Representative compounds of the invention include:

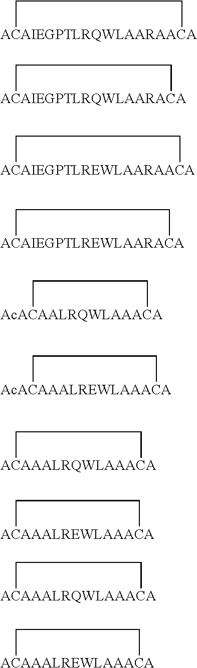

In an embodiment, in the compounds of formulae (I), (II), (III), or (IV) the amino acid residues are linked together by peptidyl linkages (amide bonds). In another embodiment, one or more of the amino acid residues are linked together by a non-peptidyl linkage, such as a —CH₂-carbamate linkage [—CH₂—OC(O)NR″—]; a phosphonate linkage; a —CH₂-sulfonamide [—CH₂—S(O)₂NR″—] linkage; a urea [—NHC(O)NH—] linkage; a —CH₂-secondary amine linkage; or an alkylated peptidyl linkage [—C(O)NR″—]; wherein R″ may be independently selected from hydrogen, optionally substituted alkyl, optionally substituted alkenyl, optionally substituted alkynyl, haloalkyl, optionally substituted cycloalkyl, optionally substituted aryl, optionally substituted arylalkyl, alkoxycarbonyl, alkylcarbonyloxy, alkylamido and alkylamino.

If present, any N-terminal amino acids in the aforementioned compounds of formula (I) each independently may be derivatised or may be an unsubstituted amine. In an embodiment, any N-terminal amino acids each independently may be —NR″₂, —NC(O)R″₂, —NR″SO₂R″, —NR″C(O)NR″₂ or —NR″C(O)OR″; wherein each R″ is as defined above.

In a further embodiment, any N-terminal amino acids may be independently a primary amine, an acetamide or a pyroglutamide; in an embodiment, a primary amine.

Any C-terminal amino acids, if present, may be a free carboxyl group or an amide. Compounds with other modifications at the C-terminus are also considered to be within the scope of the present invention.

As used herein, naturally occurring amino acids are L- or D-form of the twenty amino acids commonly found in nature. These are glycine (Gly, G), alanine (Ala, A), valine (Val, V), leucine (Leu, L), isoleucine (Ile, I), methionine (Met, M), proline (Pro, P), phenylalanine (Phe, F), tryptophan (Trp, W), serine (Ser, S), threonine (Thr, T), asparagine (Asn, N), glutamine (Gln, Q), tyrosine (Tyr, Y), cysteine (Cys, C), lysine (Lys, K), arginine (Arg, R), histidine (His, H), aspartic acid (Asp, D), and glutamic acid (Glu, E).

As used herein, non-naturally occurring amino acids include any compound with both amino and carboxyl functionality, derivatives thereof, or derivatives of a naturally occurring amino acid. These amino acids form part of the peptide chain through bonding via their amino and carboxyl groups. Alternatively, these derivatives may bond with other natural or non-naturally occurring amino acids to form a non-peptidyl linkage.

Non-naturally occurring amino acids may include amino acids that have undergone side chain modifications. Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of mixed disulfides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH. It is also possible to replace the sulphydryl groups of cysteine with selenium equivalents such that the peptide may form a diselenium bond in place of a disulfide bond, if present.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Proline residues may be modified by, for example, hydroxylation in the 4-position.

A list of some amino acids having modified side chains and some other unnatural amino acids is shown in Table A.

TABLE A

| Non-conventional | | Non-conventional | |
|---|---|---|---|
| amino acid | Code | amino acid | Code |
| L-α-aminobutyric acid | Abu | L-α-methylhistidine | Mhis |
| α-amino-α-methylbutyrate | Mgabu | L-α-methylisoleucine | Mile |
| aminocyclopropane- | Cpro | L-α-methylleucine | Mleu |
| carboxylate | | L-α-methylmethionine | Mmet |
| aminoisobutyric acid | Aib | L-α-methylnorvaline | Mnva |
| aminonorbornyl- | Norb | L-α-methylphenylalanine | Mphe |
| carboxylate | | L-α-methylserine | Mser |
| cyclohexylalanine | Chexa | L-α-methyltryptophan | Mtrp |
| cyclopentylalanine | Cpen | L-α-methylvaline | Mval |
| D-alanine | DAla | N-(N-(2,2-diphenylethyl) | Nnbhm |
| D-arginine | DArg | carbamylmethylglycine | |
| D-asparagine | DAsn | 1-carboxy-1-(2,2-diphenyl- | Nmbc |
| D-aspartic acid | DAsp | ethylamino)cyclopropane | |
| D-cysteine | DCys | L-N-methylalanine | Nmala |
| D-glutamine | DGln | L-N-methylarginine | Nmarg |
| D-glutamic acid | DGlu | L-N-methylaspartic acid | Nmasp |
| D-histidine | DHis | L-N-methylcysteine | Nmcys |
| D-isoleucine | DIle | L-N-methylglutamine | Nmgln |
| D-leucine | DLeu | L-N-methylglutamic acid | Nmglu |
| D-lysine | DLys | L-N-methylhistidine | Nmhis |
| D-methionine | DMet | L-N-methylisolleucine | Nmile |
| D-ornithine | DOrn | L-N-methylleucine | Nmleu |
| D-phenylalanine | DPhe | L-N-methyllysine | Nmlys |
| D-proline | DPro | L-N-methylmethionine | Nmmet |
| D-serine | DSer | L-N-methylnorleucine | Nmnle |
| D-threonine | DThr | L-N-methylnorvaline | Nmnva |
| D-tryptophan | DTrp | L-N-methylornithine | Nmorn |
| D-tyrosine | DTyr | L-N-methylphenylalanine | Nmphe |
| D-valine | DVal | L-N-methylproline | Nmpro |
| D-α-methylalanine | DMala | L-N-methylserine | Nmser |
| D-α-methylarginine | DMarg | L-N-methylthreonine | Nmthr |
| D-α-methylasparagine | DMasn | L-N-methyltryptophan | Nmtrp |
| D-α-methylaspartate | DMasp | L-N-methyltyrosine | Nmtyr |
| D-α-methylcysteine | DMcys | L-N-methylvaline | Nmval |
| D-α-methylglutamine | DMgln | L-N-methylethylglycine | Nmetg |
| D-α-methylhistidine | DMhis | L-N-methyl-t-butylglycine | Nmtbug |
| D-α-methylisoleucine | DMile | L-norleucine | Nle |
| D-α-methylleucine | DMleu | L-norvaline | Nva |
| D-α-methyllysine | DMlys | α-methyl-aminoisobutyrate | Maib |
| D-α-methylmethionine | DMmet | α-methyl-γ-aminobutyrate | Mgabu |
| D-α-methylornithine | DMorn | α-methylcyclohexylalanine | Mchexa |
| D-α-methylphenylalanine | DMphe | α-methylcyclopentylalanine | Mcpen |
| D-α-methylproline | DMpro | α-methyl-α-napthylalanine | Manap |
| D-α-methylserine | DMser | α-methylpenicillamine | Mpen |
| D-α-methylthreonine | DMthr | N-(4-aminobutyl)glycine | Nglu |
| D-α-methyltryptophan | DMtrp | N-(2-aminoethyl)glycine | Naeg |
| D-α-methyltyrosine | DMty | N-(3-aminopropyl)glycine | Norn |
| D-α-methylvaline | DMval | N-amino-α-methylbutyrate | Nmaabu |
| D-N-methylalanine | DNmala | α-napthylalanine | Anap |
| D-N-methylarginine | DNmarg | N-benzylglycine | Nphe |
| D-N-methylasparagine | DNmasn | N-(2-carbamylethyl)glycine | Ngln |
| D-N-methylaspartate | DNmasp | N-(carbamylmethyl)glycine | Nasn |
| D-N-methylcysteine | DNmcys | N-(2-carboxyethyl)glycine | Nglu |
| D-N-methylglutamine | DNmgln | N-(carboxymethyl)glycine | Nasp |
| γ-carboxyglutamate | Gla | N-cyclobutylglycine | Ncbut |
| 4-hydroxyproline | Hyp | N-cyclodecylglycine | Ncdec |
| 5-hydroxylysine | Hlys | N-cylcododecylglycine | Ncdod |
| 2-aminobenzoyl | Abz | N-cyclooctylglycine | Ncoct |
| (anthraniloyl) | | N-cyclopropylglycine | Ncpro |
| Cyclohexylalanine | Cha | N-cycloundecylglycine | Ncund |
| Phenylglycine | Phg | N-(2,2-diphenylethyl)glycine | Nbhm |
| 4-phenyl-phenylalanine | Bib | N-(3,3-diphenylpropyl)glycine | Nbhe |
| L-Citrulline | Cit | N-(hydroxyethyl)glycine | Nser |
| L-1,2,3,4-tetrahydroiso- | Tic | N-(imidazolylethyl))glycine | Nhis |
| quinoline-3-carboxylic acid | | N-(3-indolylyethyl)glycine | Nhtrp |
| L-Pipecolic acid (homo | Pip | N-methyl-γ-aminobutyrate | Nmgabu |
| proline) | | D-N-methylmethionine | Dnmmet |
| L-homoleucine | Hle | N-methylcyclopentylalanine | Nmcpen |
| L-Lysine (dimethyl) | DMK | D-N-methylphenylalanine | Dnmphe |
| L-Naphthylalanine | Nal | D-N-methylproline | Dnmpro |
| L-dimethyldopa or | DMD | D-N-methylthreonine | Dnmthr |
| L-dimethoxyphenylalanine | | N-(1-methylethyl)glycine | Nval |
| L-thiazolidine-4-carboxylic | THZ | N-methyla-napthylalanine | Nmanap |
| acid | | N-methylpenicillamine | Nmpen |
| L-homotyrosine | hTyr | N-(p-hydroxyphenyl)glycine | Nhtyr |
| L-3-pyridylalanine | PYA | N-(thiomethyl)glycine | Ncys |
| L-2-furylalanine | FLA | penicillamine | Pen |
| L-histidine(benzyloxymethyl) | HBO | L-α-methylalanine | Mala |
| L-histidine(3-methyl) | HME | L-α-methylasparagine | Masn |
| D-N-methylglutamate | DNmglu | L-α-methyl-t-butylglycine | Mtbug |
| D-N-methylhistidine | DNmhis | L-methylethylglycine | Metg |
| D-N-methylisoleucine | Dnmile | L-α-methylglutamate | Mglu |
| D-N-methylleucine | DNmleu | L-α-methylhomophenylalanine | Mhphe |
| D-N-methyllysine | DNmlys | N-(2-methylthioethyl)glycine | Nmet |
| N-methylcyclohexylalanine | Nmchexa | L-α-methyllysine | Mlys |
| D-N-methylornithine | DNmorn | L-α-methylnorleucine | Mnle |
| N-methylglycine | Nala | L-α-methylornithine | Morn |
| N-methylaminoisobutyrate | Nmaib | L-α-methylproline | Mpro |
| N-(1-methylpropyl)glycine | Nile | L-α-methylthreonine | Mthr |
| N-(2-methylpropyl)glycine | Nleu | L-α-methyltyrosine | Mtyr |
| D-N-methyltryptophan | DNmtrp | L-N-methylhomophenylalani | Nmhphe |
| D-N-methyltyrosine | DNmtyr | N-(N-(3,3-diphenylpropyl) | Nnbhe |
| D-N-methylvaline | DNmval | carbamylmethylglycine | |
| L-t-butylglycine | Tbug | O-methyl-L-serine | Omser |
| L-ethylglycine | Etg | O-methyl-L-homoserine | Omhser |
| L-homophenylalanine | Hphe | O-methyl-L-tyrosine | MeY |
| L-α-methylarginine | Marg | γ-aminobutyric acid | Gabu |
| L-α-methylaspartate | Masp | O-methyl-L-homotyrosine | Omhtyr |
| L-α-methylcysteine | Mcys | L-β-homolysine | BHK |
| L-α-methylglutamine | Mgln | L-ornithine | Orn |
| N-cycloheptylglycine | Nchep | N-cyclohexylglycine | Nchex |
| N-(3-guanidinopropyl)glycine | Narg | D-N-methylserine | DNmser |
| Azide containing amino acids | | | |
| Alkynyl containing amino acids | | | |

The present invention also contemplates some of the amino acids being replaced with beta amino acids, and inverse, retroinverso or similar variants of the cyclic or linear antagonists described herein. Derivatives of the present invention may also include peptoids.

Other derivatives contemplated by the present invention include a range of glycosylation variants from a completely unglycosylated molecule to a modified glycosylated molecule.

The compounds of formula (I), (II), (II), or (IV) comprise the sequence RQW (or substitutable variations thereof). It is believed that the RQW (or substitutable variations thereof) sequence plays a significant role in improving the affinity of peptidyl compounds for the thrombopoietin receptor (TPOR).

Certain compounds are also cyclic, which may decrease the rate at which the compounds degrade in biological solutions. In addition, the cyclic nature of the compounds may assist in presenting the RQW (or substitutable variations thereof) side-chains in an orientation more effective for binding to the TPOR.

Advantageously, in an embodiment, the RQW (or substitutable variations thereof) sequence is present in only one region of a peptidyl compound, such as in a compound of the formula (I), these compounds may mimic thrombopoietin but act as antagonists at the TPOR by preventing homodimerisation.

In the compounds of formula (I) the sequence RQW may be displayed, in both cases, such that the carboxyl of arginine is bound to the amino of glutamine; and such that the carboxyl of glutamine is bound to the amino of tryptophan.

As used herein, the amino acids in the sequence RQW (or substitutable variations thereof) may be either L-amino acids or D-amino acids.

The peptides according to the present invention may be prepared using standard peptide synthetic methods. For example, the peptides may be synthesised by standard solution phase methodology, as described in Hruby, Victor J.; Meyer, Jean-Philippe. Chemical synthesis of peptides. University of Arizona, USA. Editor(s): Hecht, Sidney, M. Bioorganic Chemistry: Peptides and Proteins (1998), pp 27-64, Oxford University Press, New York, N.Y.

Linear peptides may also be synthesised by solid phase methodology using Boc chemistry, as described by Schnolzer et al., 1992, Int J Pept Protein Res 40, 180-193. Following deprotection and cleavage from the solid support the reduced peptides are purified using preparative chromatography.

Linear peptides may also be synthesised by solid phase methodology using Fmoc chemistry, as described below:
1) Peptide is synthesized by Fmoc solid-phase peptide synthesis using an automatic synthesizer.
2) Peptide is synthesized from its C-terminus by stepwise addition of amino acids.
3) the first Fmoc-amino acid is attached to an insoluble support resin via an acid labile linker.
4) After deprotection of Fmoc by treatment with piperidine, the second Fmoc-amino acid is coupled utilizing a pre-activated species or in situ activation.
5) After the desired peptide is synthesized, the resin bound peptide is deprotected and detached from the resin via TFA cleavage.
6) Following deprotection and cleavage from the solid support the reduced peptides are purified using preparative chromatography.

If desired, cysteine residues in the compounds of the present invention may be oxidised in buffered systems. The oxidised peptides may then be purified using preparative chromatography. Those skilled in the art may readily determine appropriate conditions for the oxidation of the peptide.

The peptides may also be prepared using recombinant DNA technology. A nucleotide sequence encoding the desired peptide sequence may be inserted into a suitable vector and peptide expressed in an appropriate expression system.

To produce a recombinant peptide, the DNA sequence for the peptide may be obtained and then incorporated into an expression vector with an appropriate promoter. Once the expression vector is constructed, it may then be introduced into the appropriate cell line using methods including CaCl₂, CaPO₄, microinjection, electroporation, liposomal transfer, viral transfer or particle mediated gene transfer.

The host cell may comprise prokaryote, yeast or higher eukaryote cells. Suitable prokaryotes may include, but are not limited to, eubacteria, such as Gram-negative or Gram-positive organisms, including Enterobacteriaceae. Such Enterobacteriaceae may include Bacilli (e.g. B. subtilis and B. licheniformis), Escherichia (e.g. E. coli), Enterobacter, Erwinia, Klebsiella, Proteus, Pseudomonas (e.g. P. aeruginosa), Salmonella (e.g. Salmonella typhimurium), Serratia (e.g. Serratia marcescens), Shigella, and Streptomyces. Suitable eukaryotic microbes include, but are not limited to, Candida, Kluyveromyces (e.g. K. lactis, K. fragilis, K. bulgaricus, K. wickeramii, K. waltii, K. drosophilarum, K. thermotolerans and K. marxianus), Neurospora crassa, Pichia pastoris, Trichoderma reesia, Saccharomyces cerevisiae, Schizosaccharomyces pombe, Schwanniomyces (e.g. Schwanniomyces occidentalis), and filamentous fungi (e.g. Neurospora, Penicillium, Tolypocladium, and Aspergillus (e.g. A. nidulans and A. niger)) and methylotrophic yeasts (e.g. Hansenula, Candida, Kloeckera, Pichia, Saccharomyces, Torulopsis, and Rhodotorula). Suitable multicellular organisms include, but are not limited to, invertebrate cells (e.g. insect cells including Drosophila and Spodoptera), plant cells, and mammalian cell lines (e.g. Chinese hamster ovary (CHO cells), monkey kidney line, human embryonic kidney line, mouse sertoli cells, human lung cells, human liver cells and mouse mammary tumor cells). An appropriate host cell can be selected without undue experimentation by a person skilled in the art.

The cell line may then be cultured in conventional nutrient media modified for inducing promoters, selecting transformants, or amplifying the genes encoding the desired sequences. Culture conditions, such as media, temperature, pH, and the like, can be selected without undue experimentation by the person skilled in the art (for general principles, protocols and practical techniques, see Mammalian Cell Biotechnology: A Practical Approach, Butler, M. ed., IRL Press, 1991; Sambrook et al., Molecular Cloning: A Laboratory Manual, Cold Spring Harbor Laboratory Press, 1989). The cells may then be selected and assayed for the expression of the peptide using standard procedures.

Under some circumstances it may be desirable to undertake oxidative bond formation of the expressed peptide as a chemical step following peptide expression. This may be preceded by a reductive step to provide the unfolded peptide. Those skilled in the art may readily determine appropriate conditions for the reduction and oxidation of the peptide.

Cyclisation of linear peptidyl precursors of the present invention may be performed using procedures known in the art using, for instance, K₃[(FeCN)₆] or CLEAR-OX™ resin. Cyclisation of linear peptidyl precursors of the present invention may be facilitated by the incorporation of residues with particular functional groups. These may include but are not limited to a propargyl, azido or maleamide, a diaminoproprionic acid group or lysine residue. In an embodiment, the diaminoproprionic acid group or lysine residue is chloroacetylated. Cyclisation of linear peptidyl precursors of the present invention bearing reactive functional groups may be cyclised using procedures known in the art. Cyclisation of linear peptidyl precursors of the present invention may be facilitated with the addition of a reagent. In an embodiment, cyclisation of linear precursors of the present invention is facilitated with the addition of 1,2-bis(bromomethyl)benzene or 1,3-bis(bromomethyl)benzene.

In a preferred embodiment, the linear peptidyl precursor to the TPOR antagonist comprises a thiol group.

In a preferred embodiment, the linear peptidyl precursor to the TPOR antagonist comprises a propargyl group.

In a preferred embodiment, the linear peptidyl precursor to the TPOR antagonist comprises an azido group.

In a preferred embodiment, the linear peptidyl precursor to the TPOR antagonist comprises a propargyl group and an azido group.

In a preferred embodiment, the linear peptidyl precursor to the TPOR antagonist comprises a chloroacetyl group.

In a preferred embodiment, the linear peptidyl precursor to the TPOR antagonist comprises a thiol group and a chloroacetyl group.

In a preferred embodiment, the TPOR antagonist is a cyclic peptide comprising a thioether linkage.

In a preferred embodiment, the TPOR antagonist is a cyclic peptide comprising a dimethylbenzyl linkage.

In a preferred embodiment, the TPOR antagonist is a cyclic peptide comprising a triazole linkage.

In a preferred embodiment, the TPOR antagonist is a cyclic peptide comprising a thioamide linkage.

In a preferred embodiment, the TPOR antagonist is a cyclic peptide comprising a thiomaleimide linkage.

The peptides may be purified, if required, using RP-HPLC. In one embodiment the purity of the peptides of the present invention is greater than 90%, for instance greater than 91%, greater than 92%, greater than 93%, greater than 94%, greater than 95%, greater than 96%, greater than 97%, greater than 98%, or greater than 99%.

In an embodiment, it is desirable to ensure that absence or minimisation of dimeric or poly-peptide impurities which bear two or more RQW (or substitutable variations thereof) motifs. In an embodiment, such impurities are less than 2%, such as less than 1%.

In this specification unless otherwise defined “optionally substituted” means that a group may or may not be further substituted with one or more groups selected from alkyl, alkenyl, alkynyl, aryl, halo, haloalkyl, haloalkenyl, haloalkynyl, haloaryl, hydroxy, alkoxy, alkoxyamino, alkenyloxy, aryloxy, benzyloxy, haloalkoxy, haloalkenyloxy, haloaryloxy, cyano, carboxyl, nitro, amino, alkylamino, dialkylamino, alkenylamino, alkynylamino, arylamino, diarylamino, benzylamino, acyl, alkenylacyl, alkynylacyl, arylacyl, acylamino, heterocyclyl, heterocycloxy, heterocyclamino, haloheterocyclyl, carboalkoxy, carboaryloxy, alkylthio, alkylsulfonyl, alkylsulfinyl, benzylthio, sulphonamide, —C(O)NR¹R², —NR¹—C(S)NR²R³, —NR¹C(O)OR¹, —NR¹C(O)NR²R³, —NR¹C(O)R², —C(═NR¹)NR²R³, —C(═NR²R³)SR¹, —C(S)NR²R³, —C(S)NR²R³, —C(═NCN)—NR²R³, —NR¹—C(═NCN)SR¹, —NR³SO₂R¹, —NR²C(S)R¹, —NR²C(O)R¹ and —NR¹SO₂CF₃, where R¹, R² and R³ are hydrogen or lower alkyl.

Where the optional substituent includes an aromatic or heterocyclic aromatic ring, that ring may be substituted with one or more groups selected from alkyl, alkenyl, alkynyl, halo, haloalkyl, haloalkenyl, haloalkynyl, hydroxy, alkoxy and alkenyloxy.

The term “alkyl” as used alone or in combination herein refers to a straight or branched chain saturated hydrocarbon group containing from one to ten carbon atoms and the terms “C₁₋₆ alkyl” and “lower alkyl” refer to such groups containing from one to six carbon atoms, such as methyl (“Me”), ethyl (“Et”), n-propyl, isopropyl, n-butyl, isobutyl, sec-butyl, tert-butyl and the like.

“Cycloalkyl” refers to cyclic alkyl groups having a single cyclic ring or multiple condensed rings, preferably incorporating 3 to 8 carbon atoms. Such cycloalkyl groups include, by way of example, single ring structures such as cyclopropyl, cyclobutyl, cyclopentyl, cyclohexyl, cyclooctyl, and the like, or multiple ring structures such as adamantanyl, and the like.

The term “alkenyl” means a two to ten carbon, straight or branched hydrocarbon containing one or more double bonds, preferably one or two double bonds. Examples of alkenyl include ethenylene, propenylene, 1, 3-butadienyl, and 1, 3, 5-hexatrienyl.

The term “alkynyl” means a two to ten carbon, straight or branched hydrocarbon containing one or more triple bonds, preferably one or two triple bonds.

The term “alkoxycarbonyl” as used alone or in combination herein refers to a straight or branched chain alkyl group covalently bound via an —OC(O)— linkage and the terms “C₁₋₆ alkoxycarbonyl” and “lower alkoxycarbonyl” refer to such groups containing from one to six carbon atoms, such as methoxycarbonyl, ethoxycarbonyl, propoxycarbonyl, isopropoxycarbonyl, butoxycarbonyl, t-butoxycarbonyl and the like.

The term “aromatic” or “aryl” when used alone or in combination refers to an unsubstituted or optionally substituted monocyclic or bicyclic aromatic hydrocarbon ring system. Preferred aromatic ring systems are optionally substituted phenyl (“Ph”) or naphthalenyl groups.

Preferably, the aromatic or aryl group is phenyl and may be optionally substituted with up to four but more usually with one or two groups, preferably selected from C₁₋₆ alkyl, C₁₋₆ alkoxy, cyano, trifluoromethyl and halo.

The term “heteroaromatic” or “heteroaryl” group as used herein refers to a stable, aromatic monocyclic or polycyclic ring system containing carbon atoms and other atoms selected from nitrogen, sulfur and/or oxygen.

Preferably, a heteroaromatic group is a 5 or 6-membered monocyclic ring (optionally benzofused) or an 8-11 membered bicyclic ring which consists of carbon atoms and contains one, two, or three heteroatoms selected from nitrogen, oxygen and/or sulfur.

Examples of preferred heteroaromatic groups are indolyl, benzimidazole, isoxazolyl, imidazolyl, thiazolyl, isothiazolyl, pyridyl, furyl, pyrimidinyl, pyrazolyl, pyridazinyl, furazanyl and thienyl. The heteroaryl group may be attached to the parent structure through a carbon atom or through any heteroatom of the heteroaryl that results in a stable structure.

The terms “halo” and “halogen” as used herein to identify substituent moieties, represent fluorine, chlorine, bromine or iodine, preferably chlorine or fluorine.

The compounds of the present invention include compounds where one or more functional groups either on an amino acid residue side chain or the linker group are presented in their protected form. Suitable protected forms of functional groups are well known in the industry and have been described in many references such a Protecting Groups in Organic Synthesis, Greene T W, Wiley-Interscience, New York, 1981.

Protected forms may include groups which are added to enhance the solubility or other pharmacological properties of the peptides of the present invention. For instance, in one embodiment a side chain functional group of either an amino acid or a part of the Linker is protected by a hydrophilic polymer selected from poly(alkylene glycol), poly(oxyethylated polyol), poly(olefinic alcohol), poly(vinylpyrrolidone), poly(hydroxypropylmethacrylamide), poly(acrylamide), poly(N-isopropylacrylamide), poly(dimethylacrylamide), poly(hydroxyethyl(meth)acrylate), polypeptide molecules, carbohydrates, polynucleic acids, poly(acrylates), poly(poly(alkylene glycol) meth(acrylate)).

In another embodiment the protected form includes a biological recognition motif, including but not limited to, a biotin molecule, a protein or domain or fragment of a protein, an Fc domain of IgG or other antibody, a protein, a molecule (or fragment thereof), a protein G (or fragment thereof), an (oligo or poly) peptide, an (oligo or poly) nucleic acid.

The compounds used or identified according to the present invention may be in the form of a salt or pharmaceutically acceptable derivative thereof. The salts of the compounds of the invention are preferably pharmaceutically acceptable, but it will be appreciated that non-pharmaceutically acceptable salts also fall within the scope of the present invention, since these are useful as intermediates in the preparation of pharmaceutically acceptable salts or may be useful in some applications, such as probes or assays.

The term “pharmaceutically acceptable derivative” includes pharmaceutically acceptable esters, prodrugs, solvates and hydrates, and pharmaceutically acceptable addition salts of the compounds or the derivatives. Pharmaceutically acceptable derivatives may include any pharmaceutically acceptable salt, hydrate or any other compound or prodrug which, upon administration to a subject, is capable of providing (directly or indirectly) a compounds of the present invention or an active metabolite or residue thereof. “Pharmaceutically acceptable derivatives” also encompasses the protected forms of the peptides as discussed above.

The pharmaceutically acceptable salts include acid addition salts, base addition salts, salts of pharmaceutically acceptable esters and the salts of quaternary amines and pyridiniums. The acid addition salts are formed from a compound of the invention and a pharmaceutically acceptable inorganic or organic acid including but not limited to hydrochloric, hydrobromic, sulfuric, phosphoric, methanesulfonic, toluenesulphonic, benzenesulphonic, acetic, propionic, ascorbic, citric, malonic, fumaric, maleic, lactic, salicyclic, sulfamic, or tartartic acids. The counter ion of quarternary amines and pyridiniums include chloride, bromide, iodide, sulfate, phosphate, methansulfonate, citrate, acetate, malonate, fumarate, sulfamate, and tartate. The base addition salts include but are not limited to salts such as sodium, potassium, calcium, lithium, magnesium, ammonium and alkylammonium. Also, basic nitrogen-containing groups may be quaternised with such agents as lower alkyl halides, such as methyl, ethyl, propyl, and butyl chlorides, bromides and iodides; dialkyl sulfates like dimethyl and diethyl sulfate; and others. The salts may be made in a known manner, for example by treating the compound with an appropriate acid or base in the presence of a suitable solvent.

The compounds of the invention may be in crystalline form or as solvates (e.g. hydrates) and it is intended that both forms be within the scope of the present invention. The term “solvate” is a complex of variable stoichiometry formed by a solute (in this invention, a compound of the invention) and a solvent. Such solvents should not interfere with the biological activity of the solute. Solvents may be, by way of example, water, ethanol or acetic acid. Methods of solvation are generally known within the art.

The term “pro-drug” is used in its broadest sense and encompasses those derivatives that are converted in vivo to the compounds of the invention. Such derivatives would readily occur to those skilled in the art, and include, for example, compounds where a free hydroxy group is converted into an ester derivative or a ring nitrogen atom is converted to an N-oxide. Examples of ester derivatives include alkyl esters (for example acetates, lactates and glutamines), phosphate esters and those formed from amino acids (for example valine). Any compound that is a prodrug of a compound of the invention is within the scope and spirit of the invention. Conventional procedures for the preparation of suitable prodrugs according to the invention are described in text books, such as “Design of Prodrugs” Ed. H. Bundgaard, Elsevier, 1985.

The term “pharmaceutically acceptable ester” includes biologically acceptable esters of compound of the invention such as sulphonic, phosphonic and carboxylic acid derivatives.

The invention also includes where possible a salt or pharmaceutically acceptable derivative such as a pharmaceutically acceptable salt, ester, solvate and/or prodrug of the above mentioned embodiments.

In a further aspect the invention provides for the use of an effective amount of a peptidyl TPOR antagonist as defined herein (or mixture thereof) or a pharmaceutically acceptable derivative thereof, and optionally a carrier or diluent, in the manufacture of a pharmaceutical formulation (medicament) for treating a disease or condition associated with signalling via the TPO receptor.

In another aspect, the present invention provides a pharmaceutical composition for use in treating a disease or condition associated with signalling via the TPO receptor, the composition comprising an effective amount of a peptidyl TPOR antagonist as defined herein (or a mixture thereof) or a pharmaceutically acceptable derivative thereof, and optionally a carrier or diluent.

In one embodiment of the present invention the disease or condition associated with signalling via the TPO receptor is a haematological disorder, or other haematological malignancy including but not limited to myeloid proliferative disease such as chronic myeloid leukemia (‘CML’), essential thrombocythaemia, polycythemia vera, primary myelofibrosis, or acute myeloid leukaemia, or other conditions such as acute undifferentiated leukaemia, megakaryocytic leukaemia, as well as, those leukemias that have the (8;21) translocation to generate AML1-ETO acute myeloid leukemia.

By “treatment”, is meant to encompass the amelioration of symptoms of a haematological disorder as well as containing the disorder to the level of a remission.

The term “composition” is intended to include the formulation of an active ingredient with encapsulating material as carrier, to give a capsule in which the active ingredient (with or without other carrier) is surrounded by carriers.

While the compounds described herein, or a salt or a derivative thereof, may be the sole active ingredient administered to the subject, the administration of other active ingredients with the compound is within the scope of the invention. For example, the compound could be administered with one or other therapeutic agents.

As will be readily appreciated by those skilled in the art, the route of administration and the nature of the pharmaceutically acceptable carrier will depend on the nature of the condition and the mammal to be treated. It is believed that the choice of a particular carrier or delivery system, and route of administration could be readily determined by a person skilled in the art. In the preparation of any formulation containing the peptide actives care should be taken to ensure that the activity of the peptide is not destroyed in the process and that the peptide is able to reach its site of action without being destroyed. In some circumstances it may be necessary to protect the peptide by means known in the art, such as, for example, micro encapsulation. Similarly the route of administration chosen should be such that the peptide reaches its site of action.

The pharmaceutical forms suitable for injectable use include sterile injectable solutions or dispersions, and sterile powders for the extemporaneous preparation of sterile injectable solutions. They should be stable under the conditions of manufacture and storage and may be preserved against reduction or oxidation and the contaminating action of microorganisms such as bacteria or fungi.

Those skilled in the art may readily determine appropriate formulations for the compounds of the present invention using conventional approaches. Identification of preferred pH ranges and suitable excipients, for example antioxidants, is routine in the art (see for example Cleland et al, 1993). Buffer systems are routinely used to provide pH values of a desired range and include carboxylic acid buffers for example acetate, citrate, lactate and succinate. A variety of antioxidants are available for such formulations including phenolic compounds such as BHT or vitamin E, reducing agents such as methionine or sulphite, and metal chelators such as EDTA.

The solvent or dispersion medium for the injectable solution or dispersion may contain any of the conventional solvent or carrier systems for peptide actives, and may contain, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about where necessary by the inclusion of various antibacterial and antifungal agents, for example, parabens, chlorobutanol, phenol, sorbic acid, thimerosal and the like. In many cases, it will be preferable to include agents to adjust osmolality, for example, sugars or sodium chloride. In an embodiment, the formulation for injection will be isotonic with blood. Prolonged absorption of the injectable compositions can be brought about by the use in the compositions of agents delaying absorption, for example, aluminium monostearate and gelatin. Pharmaceutical forms suitable for injectable use may be delivered by any appropriate route including intravenous, intramuscular, intracerebral, intrathecal, epidural injection or infusion.

Sterile injectable solutions are prepared by incorporating the active compounds in the required amount in the appropriate solvent with various of the other ingredients such as these enumerated above, as required, followed by filtered sterilization. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients.

Other pharmaceutical forms include oral and enteral formulations of the present invention, in which the active peptide may be formulated with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal or sublingual tablets, troches, capsules, elixirs, suspensions, syrups, wafers, and the like. It will be appreciated that some of these oral formulation types, such as buccal and sublingual tablets, have the potential to avoid liver metabolism. However the compounds of the present invention may also be delivered to the stomach where liver metabolism is likely to be involved. Such compositions and preparations, in an aspect, contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations, including those that allow specific delivery of the active peptide to specific regions of the gut.

Liquid formulations may also be administered enterally via a stomach or oesophageal tube.

Enteral formulations may be prepared in the form of suppositories by mixing with appropriate bases, such as emulsifying bases or water-soluble bases. It is also possible, but not necessary, for the peptides of the present invention to be administered topically, intranasally, intravaginally, intraocularly and the like.

The present invention also extends to any other forms suitable for administration, for example topical application such as creams, lotions and gels, or compositions suitable for inhalation or intranasal delivery, for example solutions, dry powders, suspensions or emulsions. The present invention also extends to parenteral dosage forms, including those suitable for intravenous, intrathecal, and intracerebral or epidural delivery.

The compounds useful according to the present invention may be administered by inhalation in the form of an aerosol spray from a pressurised dispenser or container, which contains a propellant such as carbon dioxide gas, dichlorodifluoromethane, nitrogen, propane or other suitable gas or combination of gases. The compounds may also be administered using a nebuliser.

Pharmaceutically acceptable vehicles and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate the compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutically acceptable vehicle. The specification for the novel dosage unit forms of the invention are dictated by and directly dependent on (a) the unique characteristics of the active material and the particular therapeutic effect to be achieved, and (b) the limitations inherent in the art of compounding active materials for the treatment of disease in living subjects having a diseased condition in which bodily health is impaired as herein disclosed in detail.

As mentioned above the principal active ingredient is compounded for convenient and effective administration in effective amounts with a suitable pharmaceutically acceptable vehicle in dosage unit form. A unit dosage form can, for example, contain the principal active compound in amounts ranging from 0.25 μg to about 2000 mg. Expressed in proportions, the active compound is generally present in from about 0.25 μg to about 2000 mg/ml of carrier. In the case of compositions containing supplementary active ingredients, the dosages are determined by reference to the usual dose and manner of administration of the said ingredients.

Examples of conditions to be treated by the methods, uses and compositions of the present invention are generally those characterised by overexpression of megakaryocyte/platelet production or cells that express the TPOR and rely on TPOR signalling for their survival and proliferation. Hence the methods, uses and compositions of the present invention may be generally available for prophylactically or therapeutically treating disorders or conditions associated with signalling via the TPO receptor.

TPO receptor antagonists are potential therapeutics by virtue of their ability to bind to the TPO receptor, c-Mpl, and thus interfere with binding of TPO and thereafter inhibit subsequent cell signalling and cellular responses. TPO receptor antagonists could be used as a therapeutic in two ways.

First to inhibit the growth of malignant cells that express and utilise c-Mpl as a key receptor and signalling pathway required for cell survival and proliferation. As an example this would apply to any leukaemia where c-Mpl is expressed by the leukaemic cells and potentially contributing to the aetiology or progression of the disease. This also includes acute myeloid leukaemia at presentation or at disease relapse. This application also includes leukaemia where c-Mpl is mutated but still depends on binding of its normal ligand, TPO for cell survival and proliferation. In these situations the TPO antagonist may prevent dimerisation of c-Mpl and thus prevent downstream signalling mediating cell survival and proliferation. This effect may also induce programmed cell death of these cells and therefore actively induce killing of the malignant cells. In this aspect, this approach is used to target and kill rare leukemic stem cells within the leukemic cell population.

Another therapeutic use for TPO antagonists is based on their ability to inhibit megakaryocyte development and platelet production. In this respect the initial events leading to this outcome are identical to those described above. That is, the antagonist binds to c-Mpl, inhibiting binding of TPO but in this circumstance the outcome may not only be cell death but inhibition of megakaryocytic differentiation and subsequently, platelet production. This would be applied to treatment of some haematological malignancies, such as CML, and essential thrombocytotic and mylofibryotic disease where the platelet count reaches dangerously high levels. A potential treatment would be to give an antagonist that binds with high affinity to a single Mpl receptor but prevents dimerisation.

The peptidyl antagonist compounds of this invention may thus be used in any situation in which production of platelets or platelet precursor cells needs to be regulated, or in which the prevention of the homodimerisation (or activation) of the TPO receptor is desired. Thus, for example, the compounds of this invention may be used to treat any condition in mammal wherein there is a need to control the production of platelets, megakaryocytes, leukemic cells, and the like.

In an embodiment, the peptidyl compounds of the present invention include those which have strong binding affinities for the TPOR and may function as antagonists by preventing homodimerisation of TPO receptors. Accordingly, the present compounds are proposed to be useful for therapeutic purposes in treating a disease or condition associated with TPOR signalling, for example on platelets, megakaryocytes and other stem cells.

Therefore, from the above it can be observed that the compounds of the present invention is useful in the prevention and treatment of diseases mediated by TPO, for example haematological disorders including haematological malignancies.

As indicated above, disorders or conditions associated with signalling via the TPO receptor include but not limited to myeloproliferative disease such as chronic myeloid leukemia, essential thrombocythaemia, polycythemia vera, primary myelofibrosis, or acute myeloid leukaemia, or other conditions such as acute undifferentiated leukaemia, megakaryocytic leukaemia, as well as those leukaemias that have the (8;21) translocation to generates AML1-ETO acute myeloid leukaemia.

In an embodiment, the subject is in need of such treatment, although the compound may be administered in a prophylactic sense, such as to at risk subjects (e.g., where there is familiar history or other genetic predisposition).

For certain of the abovementioned conditions it is clear that the compounds may be used prophylactically as well as for the alleviation of acute symptoms. Accordingly, references herein to “treatment” or the like are to be understood to include such prophylactic treatment, as well as treatment of acute conditions.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Certain embodiments of the invention will now be described with reference to the following examples which are intended for the purpose of illustration only and are not intended to limit the scope of the generality hereinbefore described.

### EXAMPLES

### Example 1: Large Cyclic Peptide (LCP)

### Part A: Preparation and Characterisation of Peptides

A cyclic peptide, comprising 20 amino acid residues and with the sequence

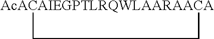

was prepared by automated Fmoc solid phase synthesis (as per Winkler, D., Riches, A., Condie, G., Tarasova, A., Andrade, J., White, J., Cablewski, T., Werkmeister, J., Haylock, D. and Meagher, L., ACS Chemical Biology 2010, DOI: 10.1021/cb100100u) by GenScript Corporation (Scotch Plains, N.J., USA). Here Ac refers to acetylation of the N-terminus of the peptide. Purification of the reduced precursor was carried out using preparative reverse phase high performance liquid chromatography (RP-HPLC) and the purity assessed using analytical RP-HPLC. Cyclisation of the peptide via the formation of an intramolecular disulphide bond was carried out by oxidation by air. Air was continuously bubbled through a dilute solution (100 mL, 0.015 μmol/mL) of the peptide in aqueous ammonia (pH 8.0) at 25° C. overnight. Aliquots were removed at various time intervals and analysed by RP-HPLC. Final purification of the cyclised product was carried out via preparative RP-HPLC and product was characterised by positive ion electrospray ionisation mass spectrometry (ESI-MS) and RP-HPLC. This peptide was received as a lyophilised powder with a purity of >95%. Subsequent analysis of the peptide as received product (LCP) by a combination of RP-HPLC, ESI-MS and Fast Protein Liquid Chromatography (FPLC) identified the presence of a dimeric contaminant which was the result of disulphide bonding between two linear AcACAIEGPTLRQWLAARAACA sequences (approx 4%). The presence of such a contaminant, could seriously compromise the antagonist activity of the “monomeric” cyclised peptide by acting as an agonist and encouraging proliferation of cells, as observed independently with other similar dimeric peptides. Therefore before biological testing of the peptide, further purification of the LCP peptide, as received, was carried out using Fast Protein Liquid Chromatography (FPLC) using a Superdex peptide column (10 mm×30 cm) in a mobile phase comprising 0.1% trifluoroacetic acid (TFA) and 10 percent acetonitrile in water. The “dimeric” contaminant (LCP-PD) was also isolated and tested with cells to confirm that it did indeed act as an agonist.

The purified monomeric, cyclic peptide product (LCP-PM) was found to have a molecular weight of 2111.60 using ESI-MS. This molecular weight corresponded to a cyclic peptide comprising one AcACAIEGPTLRQWLAARAACA sequence (20 amino acid residues) linked via one intermolecular disulphide bond (theoretical molecular weight 2112.43 a.m.u.). The purified dimeric contaminant (LCP-PD) was found to have a molecular weight of 4224.72 a.m.u. using ESI-MS. This molecular weight corresponded to a large cyclic peptide comprising two AcACAIEGPTLRQWLAARAACA sequences (40 amino acid residues) linked via two intermolecular disulphide bonds (theoretical molecular weight 4224.87 a.m.u.).

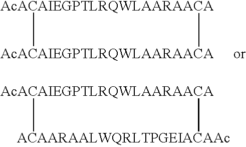

### Part B: Biological Testing of Peptides

Concentrated stock solutions of the peptides (LCP, LCP-PM and LCP-PD) were prepared in either DMSO or sterile water and further diluted into PBS to make working stock solutions for testing. The concentration range tested in most cases was 1 nM to 100 μM.

Bioactivity testing was carried out as follows. All compounds were tested on a murine TPO-dependent cell line FD-Mpl. FD-Mpl cells are derived from the haemopoietic growth factor dependent cell line FDCP-1 and express the receptor for human TPO (c-Mpl)—(as per Winkler, D., Riches, A., Condie, G., Tarasova, A., Andrade, J., White, J., Cablewski, T., Werkmeister, J., Haylock, D. and Meagher, L., ACS Chemical Biology 2010, DOI: 10.1021/cb100100u). These cells are typically cultured in media supplemented with 10% foetal bovine serum and the cytokines interleukin-3, GM-CSF or TPO (as per Naparstek, E., Pierce, J., Metcalf, D., Shadduck, R., Ihle, J., Leder, A., Sakakeeny, M. A., Wagner, K., Falco, J., Fitzgerald, T. J. and Greenberger, J. S., Blood 1986, 67, 1395-1403). In the absence of either of these factors, FDCP-1 cells rapidly undergo apoptosis (usually within 24 hours). FD-Mpl cells were maintained in DMEM medium with 10% foetal calf serum (FCS) and supplemented with 30 ng/mL of recombinant human TPO (rhTPO, Apollo Cytokine Research). For the antagonist assay, FD-Mpl cells were harvested by centrifugation and the resulting cell pellet was thoroughly washed, twice, with 10 mL PBS to remove residual cytokine. The cells were then resuspended in 5 mL of cell culture medium without rhTPO and incubated at 37° C. for 1 h (or 2 h in the case of the agonist assay; Note: short term withdrawal of rhTPO enhances up-regulation of the TPO receptor, c-Mpl). FD-Mpl cells were counted and resuspended in a mix of cell culture medium with added peptide of interest and incubated at 4° C. for 2 h (for the agonist assay this brief incubation at 4° C. for 2 h was omitted). Following this incubation, cells were supplemented with low doses of TPO (either 6 or 10 ng/mL of rhTPO) and plated at 5000 cells/well/100 μL medium in a 96-well tissue culture plate. In the case of the agonist assay the cells were not supplemented with rhTPO. The following controls were also run during the assay:
Positive control: FD-Mpl cells were cultured in the absence of peptide and supplemented with 30 ng/mL of rhTPO (Apollo Cytokine Research).
Negative control: FD-Mpl cells were cultured in the absence of peptide or rhTPO
DMSO Controls: Cells were cultured with either 0.1% or 1% DMSO in the presence or absence of 30 ng/mL of rhTPO. Note: DMSO is inhibitory to the growth of FD-Mpl cells at concentrations of 0.2% or higher.

Cells were then incubated at 37° C. for 24 hours (or 48 hours in the case of the agonist assay) and cell proliferation was assessed as follows. Cells were plated in quadruplicate onto white luminescence detection plates and cell proliferation and viability were assessed by an ATP-luminescence detection assay (CellTiter Glo Luminescent Cell Viability Assay Kit, Promega). Cells were also plated in triplicate and cell morphology was assessed using phase contrast microscopy.

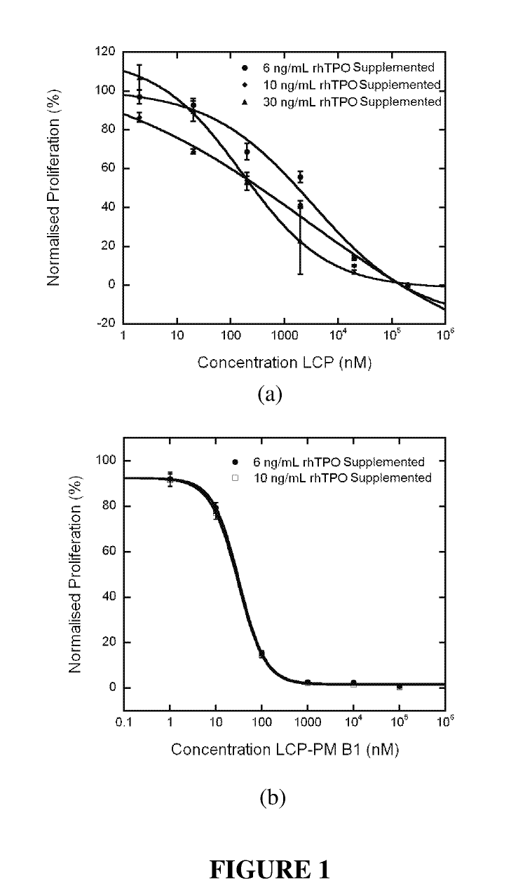

Data were analysed in the following way. For the antagonist assay, the cell numbers determined via the luminescence assay were normalised by the cell numbers obtained for FD-Mpl cells cultured in either 6 or 10 ng/mL of rhTPO without added peptide and plotted as a function of the peptide concentration (see FIGS. 1a and b). Reduction in the cell numbers as a function of increasing peptide concentration indicated competitive binding between the peptide and rhTPO at the c-Mpl receptor (i.e. the peptide is an antagonist of rhTPO). Since the peptide cannot cause dimerisation of the c-Mpl receptor resulting in activation of the receptor, and merely blocks the receptor from binding to rhTPO, the FD-Mpl cells die. For the agonist assay used with the LCP-PD peptide, the cell numbers were normalised by the cell numbers obtained with media supplemented with 30 ng/mL rhTPO and expressed as a percentage. Increasing cell numbers as a function of increasing peptide concentration (in the absence of rhTPO) indicate agonist activity for the peptide. At high concentrations, blocking of receptors with dimeric peptides, but without the dimerisation of two receptors usually occurs (i.e. the formation of 1:1 receptor:ligand complexes that inhibit the formation of the 2:1 complexes required for receptor activity), which results in declining cell proliferation.

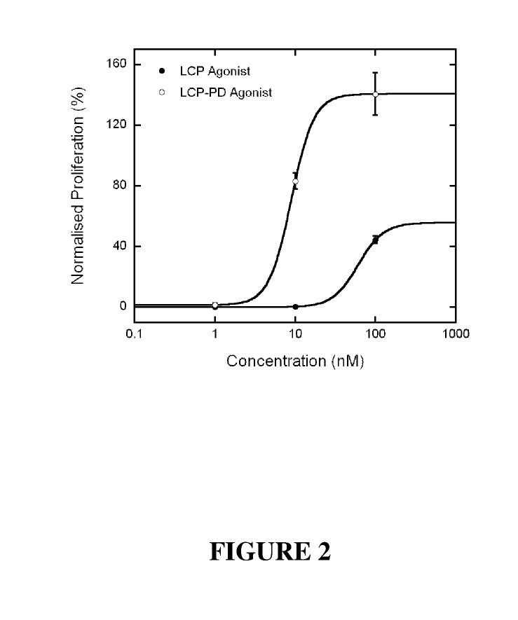

Presented in FIGS. 1 to 4 are the results obtained from the biological testing of the LCP peptide (a) before purification (LCP) and (b) after purification (LCP-PM) to remove contaminating dimer (LCP-PD). The normalised cell proliferation can be observed to decrease as a function of peptide concentration, as expected for a compound which acts as an antagonist of the c-Mpl receptor. However the concentration at which this decrease occurred was much lower in the case of LCP-PM than un-purified LCP (approximately 30 nM compared to 3 μM respectively). Clearly removing the contaminating dimer (LCP-PD) resulted in clear antagonist activity at a low peptide concentration. EC₅₀ values were estimated for the various peptides by fitting the data obtained with a standard sigmoidal shaped function using a four parameter regression analysis. Confirmation of the agonist activity of the contaminating dimeric peptide (LCP-PD) was obtained using an agonist assay. Presented in FIG. 2 is the data obtained from testing the agonist activity of both the unpurified LCP and the purified dimeric contaminant (LCP-PD). For both peptides tested in this assay the normalised cell proliferation increased as the LCP-PD concentration increased. However the concentration of unpurified LCP peptide at which increased cell proliferation was observed was much higher than that obtained for the purified dimeric contaminant LCP-PD (approximately 60 nM compared to 9 nM) and the efficacy of LCP compared to LCP-PD with respect to cell proliferation was much lower (40% compared to 140% respectively) demonstrating that the LCP-PD was a potent agonist of the c-Mpl receptor. The data presented in these two figures clearly show that without complete exclusion of dimeric contaminants from the preparation of cyclic peptides such as LCP, the biological response of cells to these peptides can be masked and indeed misinterpreted.

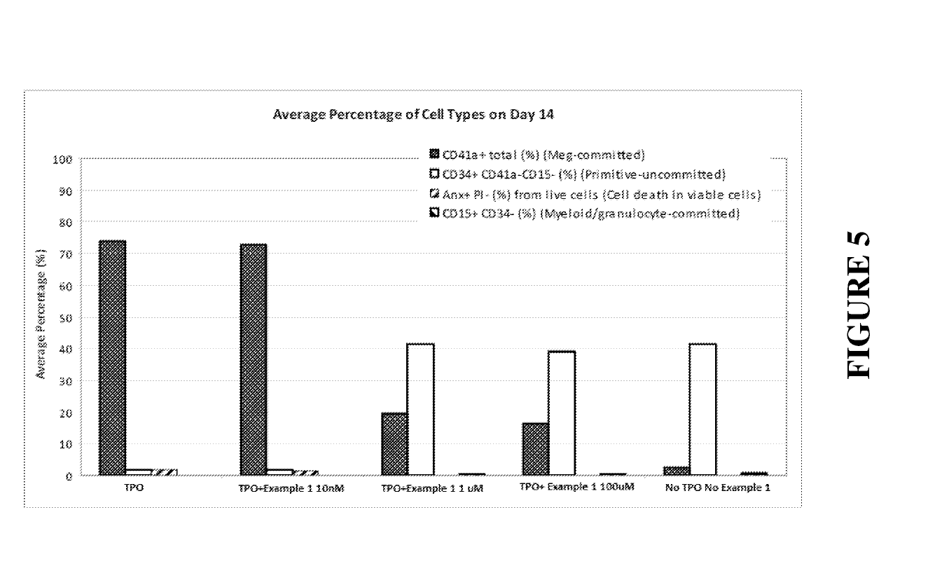

Presented in FIGS. 5 through to 16 are the results obtained from culture of human CD34+ cells isolated from cord blood. The cells are cultured in serum free media supplemented with a combination of Stem cell factor, TPO (at 100 ng/ml), Interleukin-6 and Interleukin-9 (ST69). To this combination is added increasing doses of the peptide of Example 1 and assessed total cells and their phenotype (CD34, CD41, CD15) at days 7 and 14 of culture.

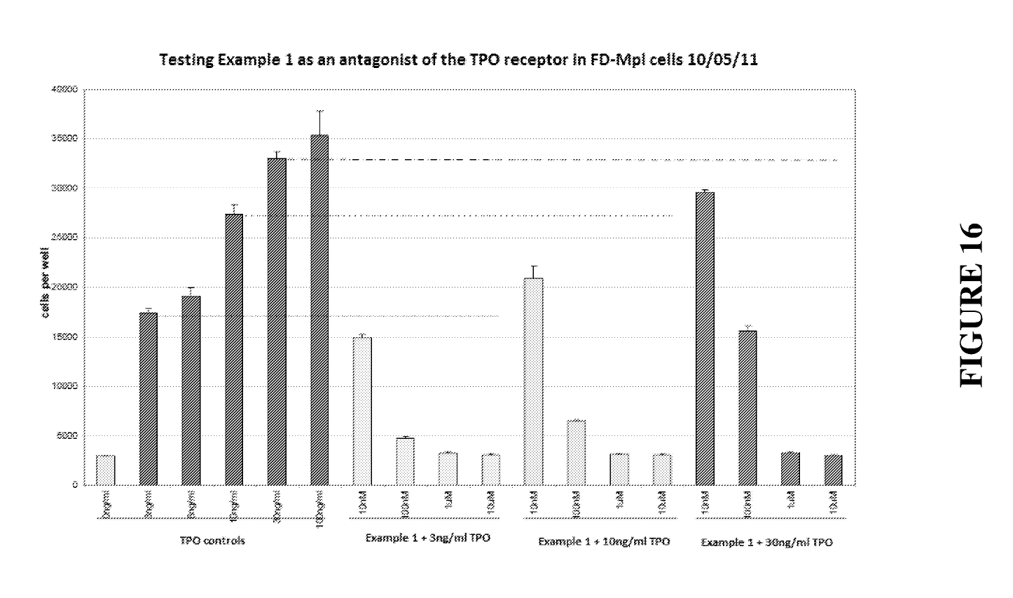

Presented in FIG. 16 are the results based on an assay of FD-mpl cells (the bioassay used to generate data within FIGS. 1-4).

### Example 2: Medium Cyclic Peptide (MCP)

### Part A: Preparation and Characterisation of Peptides

A cyclic peptide, comprising 15 amino acid residues and with the sequence

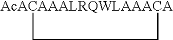

was prepared by automated Fmoc solid phase synthesis (as per Winkler, D., Riches, A., Condie, G., Tarasova, A., Andrade, J., White, J., Cablewski, T., Werkmeister, J., Haylock, D. and Meagher, L., ACS Chemical Biology 2010, DOI: 10.1021/cb100100u) by GenScript Corporation (Scotch Plains, N.J., USA). Here Ac refers to acetylation of the N-terminus of the peptide. Purification of the reduced precursor was carried using preparative reverse phase high performance liquid chromatography (RP-HPLC) and the purity assessed using analytical RP-HPLC. Cyclisation of the peptide via the formation of an intramolecular disulphide bond was carried out by oxidation by air. Air was continuously bubbled through a dilute solution (100 mL, 0.015 μmol/mL) of the peptide in aqueous ammonia (pH 8.0) at 25° C. overnight. Aliquots were removed at various time intervals and analysed by RP-HPLC. Final purification of the cyclised product was carried out via preparative RP-HPLC and product was characterised by positive ion electrospray ionisation mass spectrometry (ESI-MS) and RP-HPLC. This peptide was received as a lyophilised powder with a purity of >95%. Subsequent analysis of the as received product (MCP) by a combination of RP-HPLC, ESI-MS and Fast Protein Liquid Chromatography (FPLC) identified the presence of a dimeric contaminant which was the result of a side reaction between two AcACAAALRQWLAAACA sequences (approx 4%). The presence of such a contaminant, could seriously compromise the antagonist activity of the “monomeric” cyclised peptide by acting as an agonist and encouraging proliferation of cells. Therefore before biological testing of the peptide, further purification of the LCP peptide, as received, was carried out using Fast Protein Liquid Chromatography (FPLC) using a Superdex peptide column (10 mm×30 cm) in a mobile phase comprising 0.1% trifluoroacetic acid (TFA) and 10 percent acetonitrile in water. The “dimeric” contaminant (LCP-PD) was also isolated and tested with cells to confirm that it did indeed act as an agonist.

The purified monomeric, cyclic peptide product (MCP-PM) was found to have a molecular weight of 1529.49 using ESI-MS. This molecular weight corresponded to a cyclic peptide comprising one AcACAAALRQWLAAACA sequence (15 amino acid residues) linked via one intermolecular disulphide bond (theoretical molecular weight 1529.79 a.m.u.). The purified dimeric contaminant (MCP-PD) was found to have a molecular weight of 3059.68 a.m.u. using ESI-MS. This molecular weight corresponded to a larger cyclic peptide comprising two AcACAAALRQWLAAACA sequences (30 amino acid residues) linked via two intermolecular disulphide bonds (theoretical molecular weight 3059.57 a.m.u.).

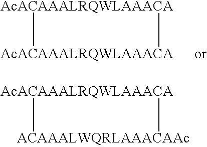

### Part B: Biological Testing of Peptide

Concentrated stock solutions of the peptides (MCP, MCP-PM and MCP-PD) were prepared in either DMSO or sterile water and further diluted into PBS to make working stock solutions for testing. The concentration range tested in most cases was 1 nM to 100 μM.

Bioactivity testing was carried out as follows. All compounds were tested on a murine TPO-dependent cell line FD-Mpl. FD-Mpl cells are derived from the haemopoietic growth factor dependent cell line FDCP-1 and express the receptor for human TPO (c-Mpl; Winkler, D., Riches, A., Condie, G., Tarasova, A., Andrade, J., White, J., Cablewski, T., Werkmeister, J., Haylock, D. and Meagher, L., ACS Chemical Biology 2010, DOI: 10.1021/cb100100u). These cells are typically cultured in media supplemented with 10% foetal bovine serum and the cytokines interleukin-3, GM-CSF or TPO (Naparstek, E., Pierce, J., Metcalf, D., Shadduck, R., Ihle, J., Leder, A., Sakakeeny, M. A., Wagner, K., Falco, J., Fitzgerald, T. J. and Greenberger, J. S., Blood 1986, 67, 1395-1403). In the absence of either of these factors, FDCP-1 cells rapidly undergo apoptosis (usually within 24 hours). FD-Mpl cells were maintained in DMEM medium with 10% foetal calf serum (FCS) and supplemented with 30 ng/mL of recombinant human TPO (rhTPO, Apollo Cytokine Research). For the antagonist assay, FD-Mpl cells were harvested by centrifugation and the resulting cell pellet was thoroughly washed, twice, with 10 mL PBS to remove residual cytokine. The cells were then resuspended in 5 mL of cell culture medium without rhTPO and incubated at 37° C. for 1 h (or 2 h in the case of the agonist assay; Note: short term withdrawal of rhTPO enhances up-regulation of the TPO receptor, c-Mpl). FD-Mpl cells were counted and resuspended in a mix of cell culture medium with added peptide of interest and incubated at 4° C. for 2 h (for the agonist assay this brief incubation at 4° C. for 2 h was omitted). Following this incubation, cells were supplemented with low doses of TPO (either 6 or 10 ng/mL of rhTPO) and plated at 5000 cells/well/100 μL medium in a 96-well tissue culture plate. In the case of the agonist assay the cells were not supplemented with rhTPO. The following controls were also run during the assay:
Positive control: FD-Mpl cells were cultured in the absence of peptide and supplemented with 30 ng/mL of rhTPO (Apollo).
Negative control: FD-Mpl cells were cultured in the absence of peptide or rhTPO
DMSO Controls: Cells were cultured with either 0.1% or 1% DMSO in the presence or absence of 30 ng/mL of rhTPO. Note: DMSO is inhibitory to the growth of FD-Mpl cells at concentrations of 0.2% or higher.

Cells were then incubated at 37° C. for 24 hours (or 48 hours in the case of the agonist assay) and cell proliferation was assessed as follows. Cells were plated in quadruplicate onto white luminescence detection plates and cell proliferation and viability were assessed by an ATP-luminescence detection assay (CellTiter Glo Luminescent Cell Viability Assay Kit, Promega). Cells were also plated in triplicate and cell morphology was assessed using phase contrast microscopy.

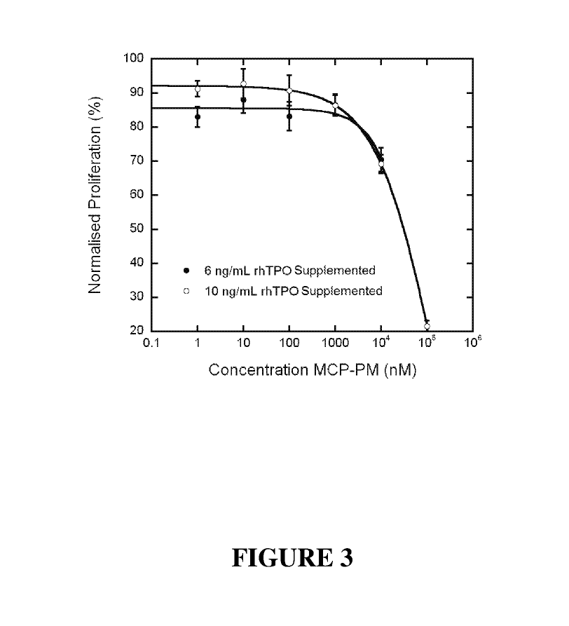

Data were analysed in the following way. For the antagonist assay, the cell numbers determined via the luminescence assay were normalised by the cell numbers obtained for FD-Mpl cells cultured in either 6 or 10 ng/mL of rhTPO without added peptide and plotted as a function of the peptide concentration (see FIG. 3). Reduction in the cell numbers as a function of increasing peptide concentration indicated competitive binding between the peptide and rhTPO at the c-Mpl receptor (i.e. the peptide is an antagonist of rhTPO). Since the peptide cannot cause dimerisation of the c-Mpl receptor resulting in activation of the receptor, and merely blocks the receptor from binding to rhTPO, the FD-Mpl cells die. For the agonist assay used with the MCP-PD peptide, the cell numbers were normalised by the cell numbers obtained with media supplemented with 30 ng/mL rhTPO and expressed as a percentage. Increasing cell numbers as a function of increasing peptide concentration (in the absence of rhTPO) indicate agonist activity for the peptide. At high concentrations, blocking of receptors with dimeric peptides, but without the dimerisation of two receptors usually occurs (i.e., the formation of 1:1 receptor: ligand complexes that inhibit the formation of the 2:1 complexes required for receptor activity), which results in declining cell proliferation.

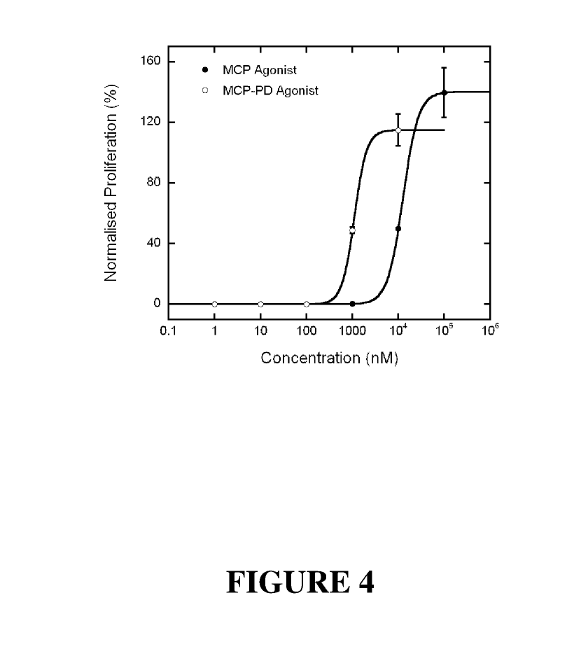

Presented in FIG. 3 are the results obtained from the biological testing of the MCP-PM peptide, i.e. after purification to remove contaminating dimer (MCP-PD). The normalised cell proliferation was observed to decrease as a function of peptide concentration, as expected for a compound which acts as an antagonist of the c-Mpl receptor. However the concentration at which this decrease occurred was higher than in the case of LCP-PM (approximately 20-30 μM compared to 30 nM respectively, see FIG. 1b), suggesting that the MCP-PM was a much weaker antagonist of rhTPO than LCP-PM. Note that the MCP-PM EC₅₀ value was an estimate only as even at the highest concentration tested (100 μM), the cell numbers did not fall to zero. Clearly removing the contaminating dimer (MCP-PD) resulted in clear antagonist activity at a moderately high peptide concentration. EC₅₀ values were estimated for the various peptides by fitting the data obtained with a standard sigmoidal shaped function using a four parameter regression analysis. Confirmation of the agonist activity of the contaminating dimeric peptide (MCP-PD) was obtained using an agonist assay. Presented in FIG. 4 are the data obtained from testing the agonist activity of both the unpurified MCP and the purified dimeric contaminant (MCP-PD). For both peptides tested in this assay the normalised cell proliferation increased as the MCP-PD concentration increased. However the concentration of unpurified MCP peptide at which increased cell proliferation was observed was much higher than that obtained for the purified dimeric contaminant MCP-PD (approximately 12.6 μM compared to 1.1 μM) demonstrating that the MCP-PD was a reasonably potent agonist of the c-Mpl receptor. The data presented in these two figures clearly show that without complete exclusion of dimeric contaminants from the preparation of cyclic peptides such as MCP, the biological response of cells to these peptides can be masked and indeed misinterpreted.

### Examples 3-11 Preparation of Cysteine-Cysteine Linked Cyclic Peptides

### Part A: Preparation and Characterisation of Peptides

The cyclic peptides displayed in Table 2 below were prepared as per Examples 1 and 2.

### Part B: Biological Testing of Peptides

The results (antagonism activity as IC50 (nM) displayed in Table 2 below were obtained as per the protocols of Examples 1 and 2.

TABLE 2
Antagonism of cyclic peptides

| Peptide | Sequence | IC50 (nM) |
|---|---|---|
| Example 1 | 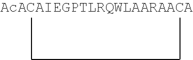 | <100 |
| Example 3 | 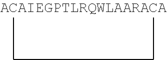 | <100 |
| Example 4 | 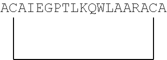 | 100-300 |
| Example 5 | 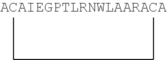 | NR* |
| Example 6 | 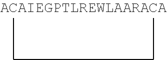 | 100-300 |
| Example 7 | 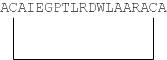 | 300-1500 |
| Example 8 | 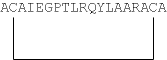 | 300-1500 |
| Example 9 | 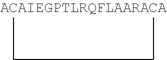 | 300-1500 |
| Example 10 | 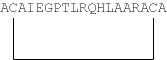 | >1500 |
| Example 11 | 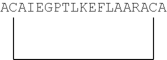 | NR* |

*Not Recorded

### Examples 12 to 17—Synthesis of Linear Peptides

The following peptides were produced by state-of-the-art methods of solid phase peptide synthesis using Fmoc/t-Bu chemistry [Int. J. Pept. Protein Res. 1990, 35, 161] on RinkAmide ChemMatrix resin. The standard synthesis procedure involved as the main steps (i) deprotection of the N^{α}-Fmoc protecting group with 20% piperidine in DMF, (ii) coupling mediated by mixtures of coupling reagents TBTU/HOBt/DIEA in DMF, and washes.

The linear peptides in Table 3 were prepared according to the procedure described above:

TABLE 3

| | | MW_{calc} | MW_{found} |
|---|---|---|---|
| Example 12 | Ac-ACAIEGPTLRQWLAARAACA-NH₂ | 2112.1 | 2113.08 |
| Example 13 | Ac-A-Aza-AIEGPTLRQWLAARAA-Pra-A-NH₂ | 2113.5 | 2113.35 |
| Example 14 | Ac-A-Dap(ClAc)-AIEGPTERQWLAARAACA-NH₂ | 2171.1 | 2171.7 |
| Example 15 | Ac-A-Lys(ClAc)-AIEGPTLRQWLAARAACA-NH₂ | 2213.1 | 2213.4 |
| Example 16 | Ac-A-Dap(Mal)-ATEGPTERQWLAARAACA-NH₂ | 2246.2 | 2247.15 |
| Example 17 | Ac-A-Lys(Mal)-AIEGPTLRQWLAARAACA-NH₂ | 2288.8 | 2288.3 |

For Example 13, the cysteine residues at positions 2 and 19 of the sequence in Example 12 were replaced with 3-azido-L-alanine and L-propargylglycine respectively.

For Examples 14 and 16, the cysteine residue at position 2 of the sequence in Example 12 was replaced with L-2,3-diaminoproprionic acid (Dap).

For Examples 15 and 17, the cysteine residue at position 2 of the sequence in Example 12 was replaced with a lysine residue.

For the synthesis of Examples 14 to 17, the β-amino group of Dap and the ε-amino group of lysine was protected with a methyltrityl (Mtt) group prior to incorporation into the peptide sequence. After the desired peptide sequence was reached, the Mtt protecting group was removed from the amino group using 1,8% TFA in CH₂Cl₂, allowing selective removal in the presence of other protecting groups which require up to 95% TFA for removal [J. Pep. Res. 2002, 60, 300]. The β-amino group of Dap and the ε-amino group of lysine was then further modified with either a chloroacetyl (ClAc) or a maleimide (Mal) group. Functionalization of the amino group of the side chain was carried out on resin by treatment with 5-fold molar excess of chloroacetic acid or N-maleoyl-β-alanine and DIPCI (diisopropylcarbodiimide) (1:1) in CH₂Cl₂.

The linear peptides of Examples 12 to 17 were obtained after full deprotection and cleavage of the respective peptide resins using a CF₃COOH/H₂O/triisopropilsilane (95:2.5:2.5) cleaving mixture. Prior to cyclisation, peptides were precipitated by addition of chilled diethyl ether, taken up in aqueous CH₃COOH (10% v/v) and lyophilized. Reverse-phase HPLC purification gave homogenous materials with the expected mass (ESI MS).

### MS Characterization

All peptides were purified with preparative HPLC and characterised with analytical HPLC as follows:

Linear peptides: LC MS 2020 Shimadzu
Cyclic peptides: Thermo Q Exactive

| HPLC | Method | Column |
|---|---|---|
| Analytical | 25-45% ACN in 15 min | Phenomenex Luna C8, 5 u, |
| | | 100 A, 100 × 4.6 mm |
| Preparative | 25-45% ACN in 20 min | Phenomenex Luna C8, 5 u, |
| | | 100 A, 250 × 10 mm |

Synthesis of Cyclic Peptides from Linear Peptides

Cyclic peptides were synthesised as described above. A low peptide concentration was used to avoid dimer formation (1-2 mg peptide/10 mL of buffer [50-100 μM]).

### Example 18

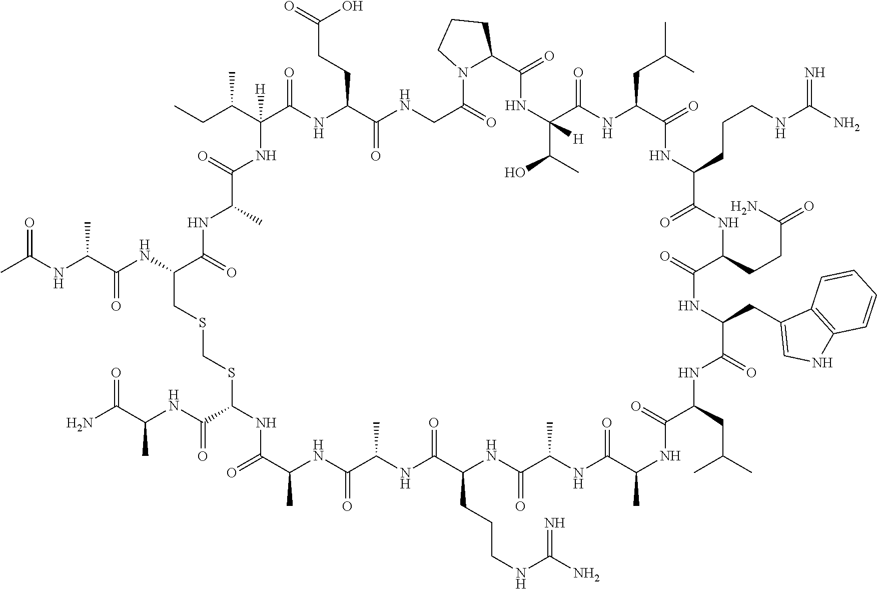 [SMILES: CC[C@H](C)[C@@H]1NC(=O)[C@H](C)NC(=O)[C@@H](NC(=O)[C@@H](C)NC(C)=O)CSCS[C@@H](C(=O)N[C@@H](C)C(N)=O)NC(=O)[C@H](C)NC(=O)[C@H](C)NC(=O)[C@H](CCCNC(=N)N)NC(=O)[C@H](C)NC(=O)[C@H](C)NC(=O)[C@H](CC(C)C)NC(=O)[C@H](Cc2c[nH]c3ccccc23)NC(=O)[C@H](CCC(N)=O)NC(=O)[C@H](CCCNC(=N)N)NC(=O)[C@H](CC(C)C)NC(=O)[C@H]([C@@H](C)O)NC(=O)[C@@H]2CCCN2C(=O)CNC(=O)[C@H](CCC(=O)O)NC1=O]

25 mg of the peptide of Example 12 was dissolved in 250 mL of water and left exposed to air. Air-oxidation reaction is very slow and was completed after 270 h. Progress of the reaction was monitored by HPLC (the retention times of linear and cyclic LCP4 peptide are different—cyclic construct is eluted ˜1 min before linear) and LC MS. Cyclic peptide was purified by preparative RP-HPLC. Final product (LCP4 cyclic) was satisfactory characterized for purity and identity by analytical HPLC and ESI MS, respectively.

### Example 19

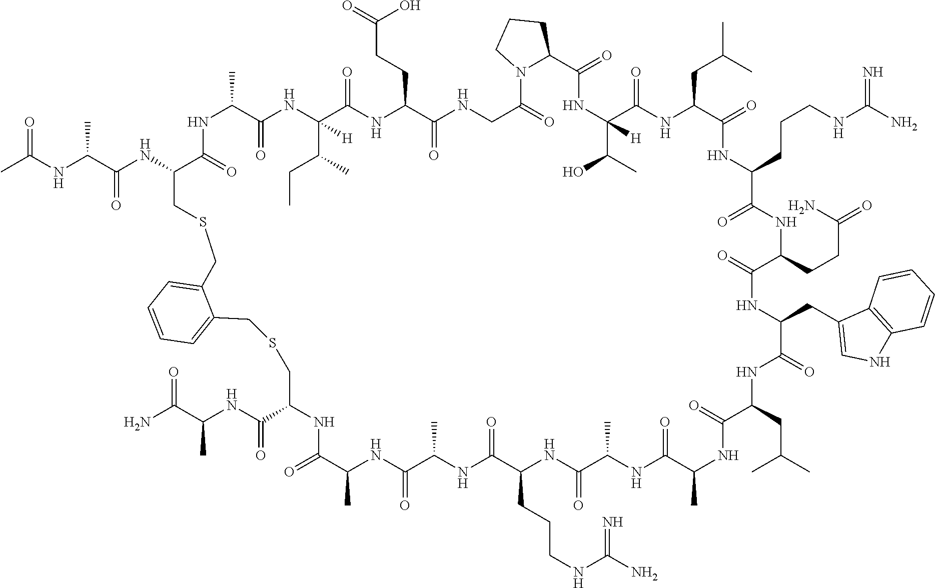 [SMILES: CC[C@@H](C)[C@H]1NC(=O)[C@@H](C)NC(=O)[C@@H](NC(=O)[C@@H](C)NC(C)=O)CSCc2ccccc2CSC[C@@H](C(=O)N[C@@H](C)C(N)=O)NC(=O)[C@H](C)NC(=O)[C@H](C)NC(=O)[C@H](CCCNC(=N)N)NC(=O)[C@H](C)NC(=O)[C@H](C)NC(=O)[C@H](CC(C)C)NC(=O)[C@H](Cc2c[nH]c3ccccc23)NC(=O)[C@H](CCC(N)=O)NC(=O)[C@H](CCCNC(=N)N)NC(=O)[C@H](CC(C)C)NC(=O)[C@H]([C@@H](C)O)NC(=O)[C@@H]2CCCN2C(=O)CNC(=O)[C@H](CCC(=O)O)NC1=O]

8 mg (˜4 μmol) of the peptide of Example 12 was dissolved in 40 ml of 0.05M phosphate buffer, pH 7.4; leq of 1,2-DBMB (1,2-bis(bromomethyl)benzene) was added to the peptide solution [ACS Chem. Biol. 2012, 7, 817]. Reaction mixture was left ON at 30° C. The progress of the reaction was monitored by HPLC and LC MS. Purification and characterization of the final product as above.

### Example 20

The cyclic peptide was prepared according to the procedure for Example 19, except 1,3-DBMB (1,3-bis(bromomethyl)benzene) was used instead.

### Example 21

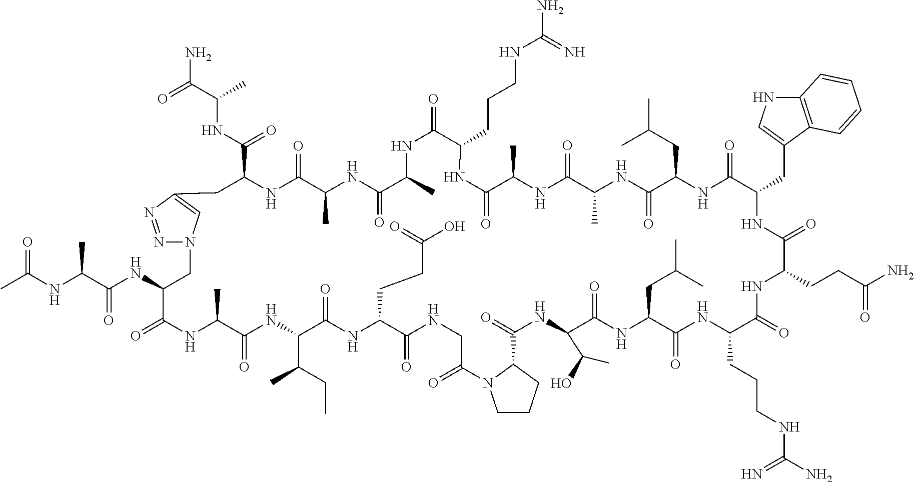 [SMILES: CC[C@@H](C)[C@@H]1NC(=O)[C@H](C)NC(=O)[C@@H](NC(=O)[C@H](C)NC(C)=O)Cn2cc(nn2)C[C@@H](C(=O)N[C@@H](C)C(N)=O)NC(=O)[C@H](C)NC(=O)[C@H](C)NC(=O)[C@H](CCCNC(=N)N)NC(=O)[C@@H](C)NC(=O)[C@@H](C)NC(=O)[C@@H](CC(C)C)NC(=O)[C@H](Cc2c[nH]c3ccccc23)NC(=O)[C@H](CCC(N)=O)NC(=O)[C@H](CCCNC(=N)N)NC(=O)[C@H](CC(C)C)NC(=O)[C@@H]([C@@H](C)O)NC(=O)[C@@H]2CCCN2C(=O)CNC(=O)[C@@H](CCC(=O)O)NC1=O]

8 mg (˜4 μmol) of the peptide in Example 13 was dissolved in 40 ml of tBuOH/H₂O (1:1); 17.62 mg of ascorbic acid (100 μmol) and 25 mg of CuSO₄.5H₂O (100 μmol) was added to the peptide solution [J. Pept. Sci. 2009, 15, 451]. Reaction mixture was left for 6 h at 30° C. The progress of the reaction was monitored by HPLC and LC MS. Purification and characterization of the final product as above.

### Example 21

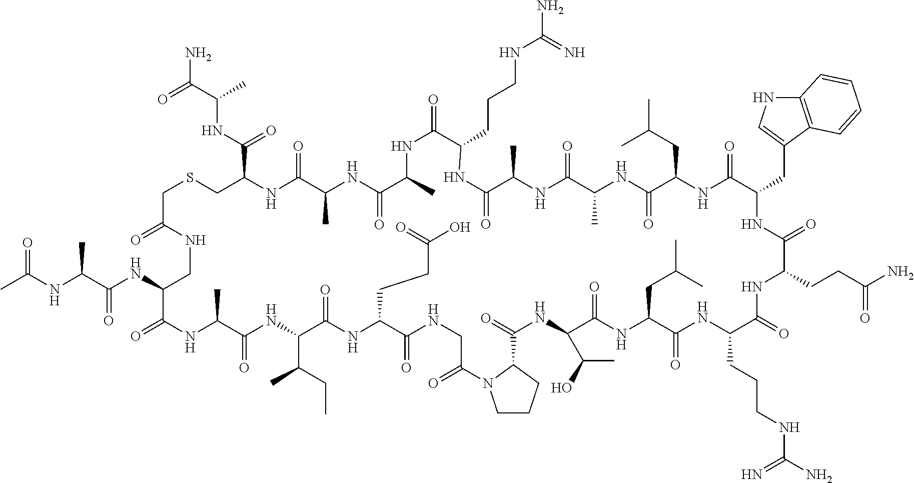 [SMILES: CC[C@@H](C)[C@@H]1NC(=O)[C@H](C)NC(=O)[C@@H](NC(=O)[C@H](C)NC(C)=O)CNC(=O)CSC[C@@H](C(=O)N[C@@H](C)C(N)=O)NC(=O)[C@H](C)NC(=O)[C@H](C)NC(=O)[C@H](CCCNC(=N)N)NC(=O)[C@@H](C)NC(=O)[C@@H](C)NC(=O)[C@@H](CC(C)C)NC(=O)[C@H](Cc2c[nH]c3ccccc23)NC(=O)[C@H](CCC(N)=O)NC(=O)[C@H](CCCNC(=N)N)NC(=O)[C@H](CC(C)C)NC(=O)[C@@H]([C@@H](C)O)NC(=O)[C@@H]2CCCN2C(=O)CNC(=O)[C@@H](CCC(=O)O)NC1=O]

For conjugation, 3 mg of the peptide of Example 14 was dissolved in 30 mL of 50 mM phosphate buffer, pH 7.4, 40° C. [adapted from: Bioconjugate Chem. 2013, 24, 578]. The conjugation was monitored by HPLC and LC MS at 1 h intervals, the retention times of linear and cyclic version are different cyclic construct is eluted ˜1.5 min before linear. When no changes in the HPLC profile was observed, the reaction was stopped with 3 mL of CH₃COOH. Purification and characterization as above.

### Example 22

The cyclic peptide was synthesised as for Example 21, except for the use of the peptide of Example 15 as starting material.

### Example 23

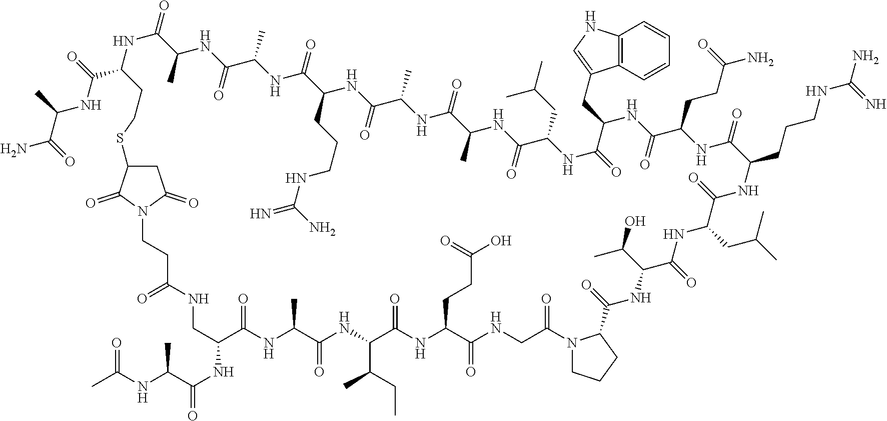 [SMILES: CC[C@@H](C)[C@@H]1NC(=O)[C@H](C)NC(=O)[C@H](NC(=O)[C@H](C)NC(C)=O)CNC(=O)CCN2C(=O)CC(SCC[C@H](C(=O)N[C@H](C)C(N)=O)NC(=O)[C@H](C)NC(=O)[C@H](C)NC(=O)[C@H](CCCNC(=N)N)NC(=O)[C@H](C)NC(=O)[C@H](C)NC(=O)[C@H](CC(C)C)NC(=O)[C@@H](Cc3c[nH]c4ccccc34)NC(=O)[C@@H](CCC(N)=O)NC(=O)[C@@H](CCCNC(=N)N)NC(=O)[C@H](CC(C)C)NC(=O)[C@@H]([C@@H](C)O)NC(=O)[C@@H]3CCCN3C(=O)CNC(=O)[C@H](CCC(=O)O)NC1=O)C2=O]

For conjugation, the peptide of Example 16 were dissolved in H₂O, RT (adapted from: Bioconjugate Chem. 2013, 24, 578). HPLC analysis of an aliquot showed reaction to be complete within minutes. Purification and characterization as above.

### Example 24

The cyclic peptide was synthesised as for Example 23, except for the use of the peptide of Example 17 as starting material.

| | | MW_{calc} | MW_{found} |
|---|---|---|---|
| Example 18 | 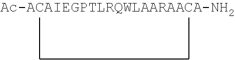 | 2110.1 | 2111.06 |
| Example 19 | 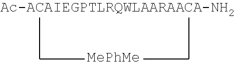 | 2214.1 | 2215.13 |
| Example 20 | 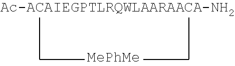 | 2214.1 | 2215.13 |
| Example 21 | 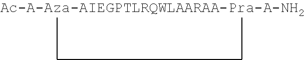 | 2113.5 | 2113.35 |
| Example 22 | 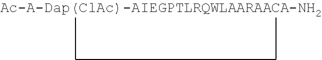 | 2135.1 | 2136.12 |
| Example 23 | 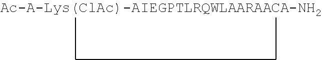 | 2177.1 | 2178.17 |
| Example 24 | 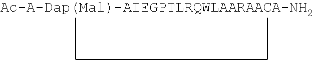 | 2246.2 | 2247.15 |
| Example 25 | 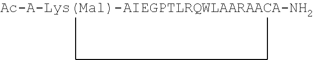 | 2288.2 | 2288.19 |

1. A method of treating a disease or condition associated with signalling via the TPO receptor, said method comprising the step of administering an effective amount of a peptidyl TPO receptor antagonist to a subject in need thereof, wherein the peptidyl TPOR receptor antagonist is a cyclic or linear peptidyl compound comprising the following structural formula (I):
X_{bb}-Xₐₐ-X_{cc} formula (I)
wherein X_{bb} represents a residue of an amino acid selected from arginine (R) and lysine (K);
Xₐₐ represents a residue of an amino acid selected from glutamine (Q), asparagine (N), aspartic acid (D), and glutamic acid (E);
X_{cc} represents a residue of an amino acid selected from tryptophan (W), phenylalanine (F), tyrosine (Y), and histidine (H);
or a salt thereof.

2. The method according to claim 1 wherein the peptidyl TPOR antagonist is a cyclic or linear peptidyl compound comprising the structural formula (Ia):
L-X_{bb}-Xₐₐ-X_{cc}-L formula (Ia)
wherein Xbb represents a residue of an amino acid selected from arginine (R) and lysine (K);
Xaa represents a residue of an amino acid selected from glutamine (Q), asparagine (N), aspartic acid (D), and glutamic acid (E);
Xcc represents a residue of an amino acid selected from tryptophan (W), phenylalanine (F), tyrosine (Y), and histidine (H);
or a salt thereof.

3. The method according to claim 1 wherein the peptidyl TPOR antagonist is a cyclic or linear peptidyl compound comprising the structural formula (Ib):
IEGPTL-X_{bb}-Xₐₐ-X_{cc}-L formula (Ib)
wherein Xbb represents a residue of an amino acid selected from arginine (R) and lysine (K);
Xaa represents a residue of an amino acid selected from glutamine (Q), asparagine (N), aspartic acid (D), and glutamic acid (E);
Xcc represents a residue of an amino acid selected from tryptophan (W), phenylalanine (F), tyrosine (Y), and histidine (H);
or a salt thereof.

4. The method according to claim 1 wherein the peptidyl TPOR antagonist is a cyclic or linear peptidyl compound comprising the structural formula (Ic):
PTL-X_{bb}-Xₐₐ-X_{cc}-LAARA formula (Ic)
wherein Xbb represents a residue of an amino acid selected from arginine (R) and lysine (K);
Xaa represents a residue of an amino acid selected from glutamine (Q), asparagine (N), aspartic acid (D), and glutamic acid (E);
Xcc represents a residue of an amino acid selected from tryptophan (W), phenylalanine (F), tyrosine (Y), and histidine (H);
or a salt thereof.

5. The method according to claim 1 wherein the peptidyl TPOR antagonist is a cyclic or linear peptidyl compound comprising the structural formula (Id):
IEGPTL-X_{bb}-Xₐₐ-X_{cc}-LAARA formula (Id)
wherein Xbb represents a residue of an amino acid selected from arginine (R) and lysine (K);
Xaa represents a residue of an amino acid selected from glutamine (Q), asparagine (N), aspartic acid (D), and glutamic acid (E);
Xcc represents a residue of an amino acid selected from tryptophan (W), phenylalanine (F), tyrosine (Y), and histidine (H);
or a salt thereof.

6. The method according to claim 1 wherein Xbb is arginine (R).

7. The method according to claim 1 wherein Xaa is glutamine (Q).

8. The method according to claim 1 wherein Xcc is tryptophan (W).

9. The method according to claim 1 wherein the peptidyl TPOR is cyclic.

10. (canceled)

11. The method according to claim 1 wherein the TPOR antagonist is a cyclic peptidyl compound comprising the formula (II): 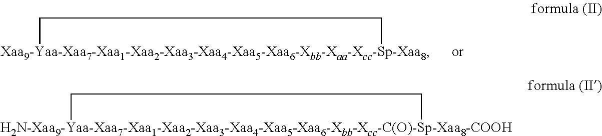
wherein Xaa1, Xaa2, Xaa3, Xaa4, Xaa5, Xaa6, Xaa7, Xaa8, and Xaa9 are each independently a residue of any naturally or non-naturally occurring amino acid or are absent;
Xbb represents a residue of an amino acid selected from arginine (R) and lysine (K);
Xaa represents a residue of an amino acid selected from glutamine (Q), asparagine (N), aspartic acid (D) and glutamic acid (E);
Xcc represents a residue of an amino acid selected from tryptophan (W), phenylalanine (F), tyrosine (Y), and histidine (H);
Yaa represents a residue of a natural or non-naturally occurring amino acid which is linked to Sp;
Sp represents an amino acid spacer of 3-30 residues in length selected from naturally and non-naturally occurring amino acids which is linked to Yaa;
or a salt or protected form thereof.

12. (canceled)

13. The method according to claim 10 wherein
Xaa1 is Gly, Ala, Val, Leu, Ile, Met, Pro or Phe;
Xaa2 is Ser, Thr, Asn, Gin, Tyr, Lys, Arg, His, Asp or Glu;
Xaa3 is Gly, Ala, Val, Leu, Ile, Pro or Phe;
Xaa4 is Ala, Val, Leu, Ile, Pro or Phe;
Xaa5 is Ser, Thr, Asn, Gin, Tyr, Cys, Asp or Glu;
Xaa6 is Gly, Ala, Val, Leu, Ile, Met, Pro or Phe; and
Xaa7 is Gly.

14. The method according to claim 10 wherein
Xaa1 is Ile;
Xaa2 is Glu;
Xaa3 is Gly;
Xaa4 is Pro;
Xaa5 is Thr;
Xaa6 is Leu; and
Xaa7 is Gly.

15. The method according to claim 10 wherein Xaa8 and Xaa9 are independently A or absent.

16. (canceled)

17. The method according to claim 1 wherein the peptidyl TPOR antagonist is a cyclic or linear peptidyl compound comprising the structural formula (IIIa):
LRQWL formula (IIIa)
or a salt thereof.

18. The method according to claim 1 wherein the peptidyl TPOR antagonist is a cyclic or linear peptidyl compound comprising the structural formula (IIIb):
IEGPTLRQWL formula (IIIb)
or a salt thereof.

19. The method according to claim 1 wherein the peptidyl TPOR antagonist is a cyclic or linear peptidyl compound comprising the structural formula (IIIc):
PTLRQWLAARA formula (IIIc)
or a salt thereof.

20. The method according to claim 1 wherein the peptidyl TPOR antagonist is a cyclic or linear peptidyl compound comprising the structural formula (IIId):
IEGPTLRQLAARA formula (IIId)
or a salt thereof.

21. The method according to claim 1 wherein the peptidyl TPOR antagonist is:

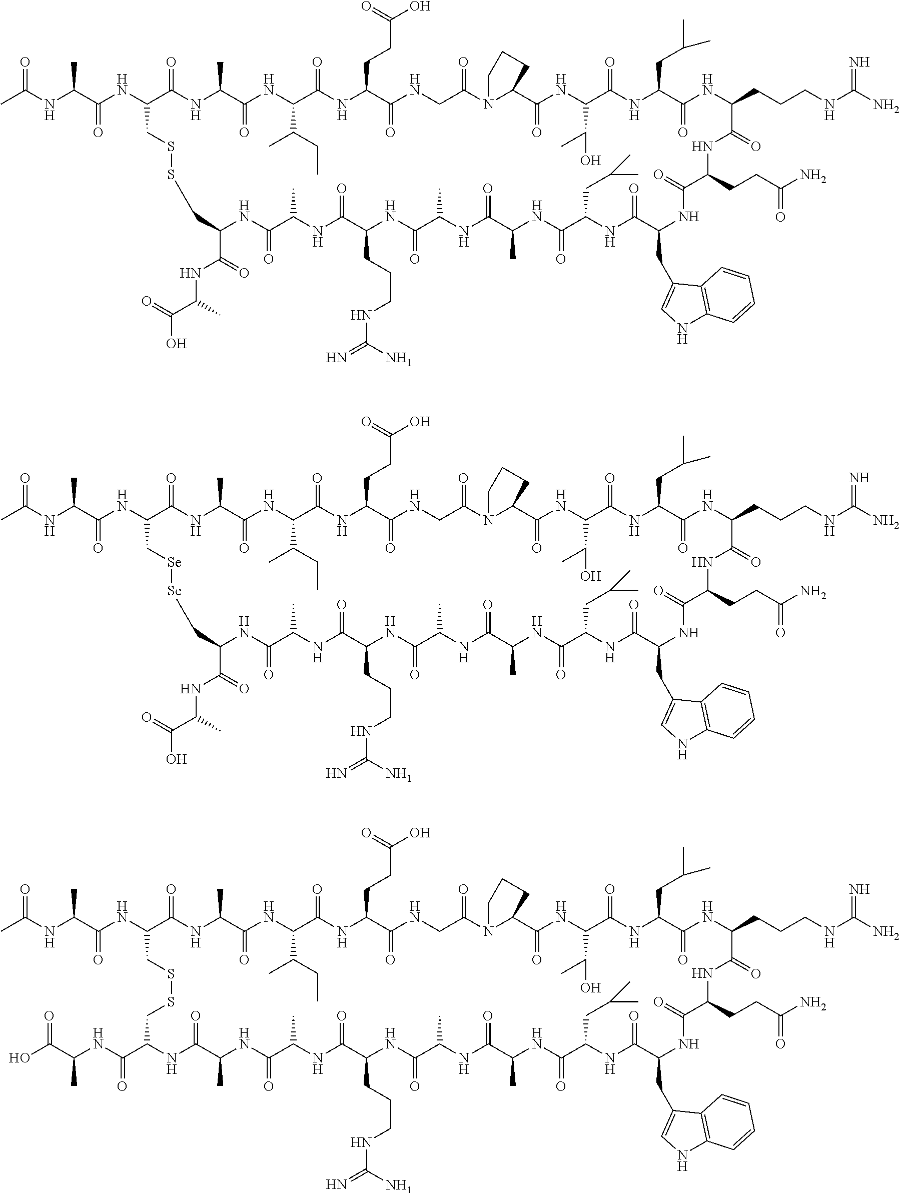

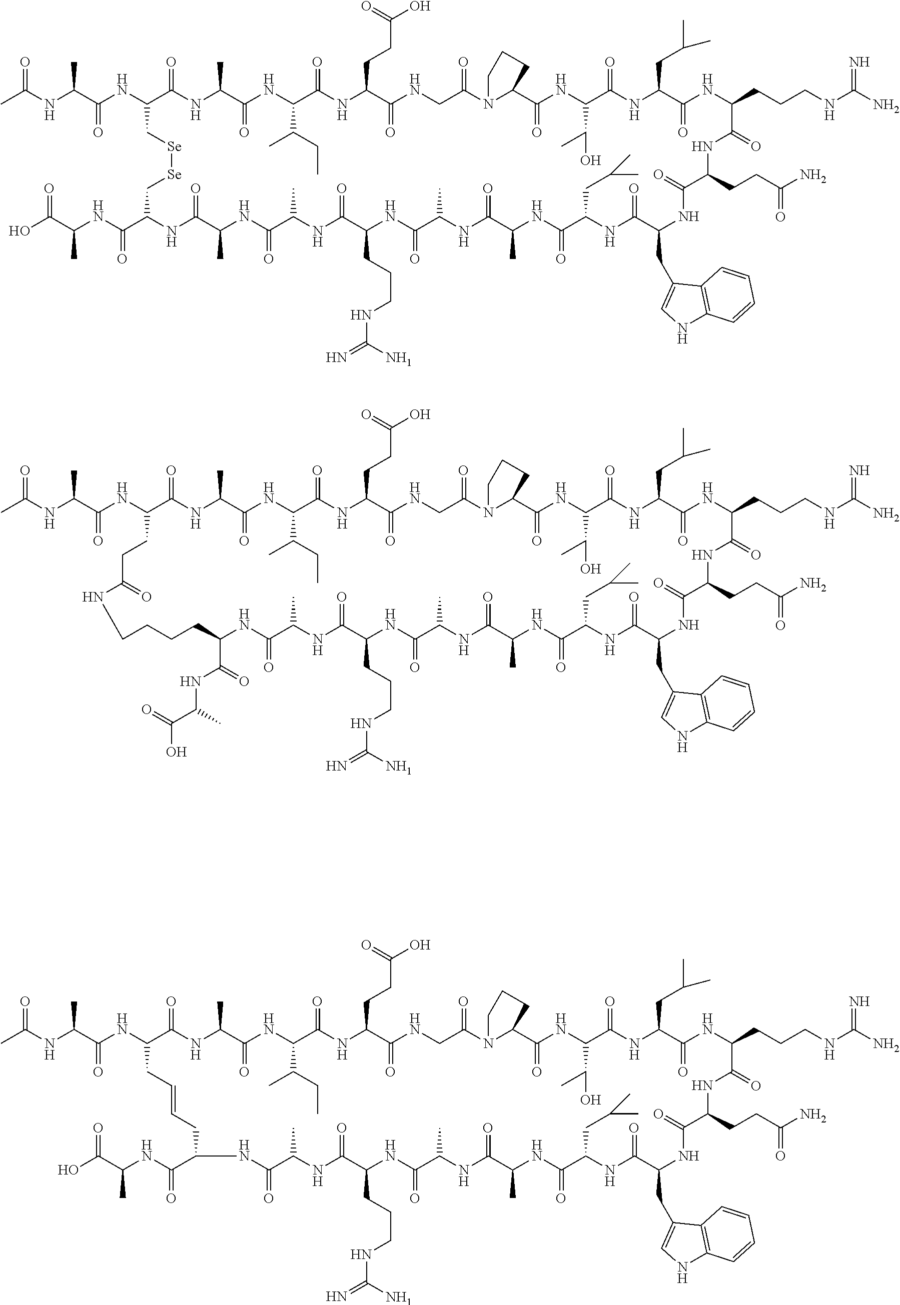

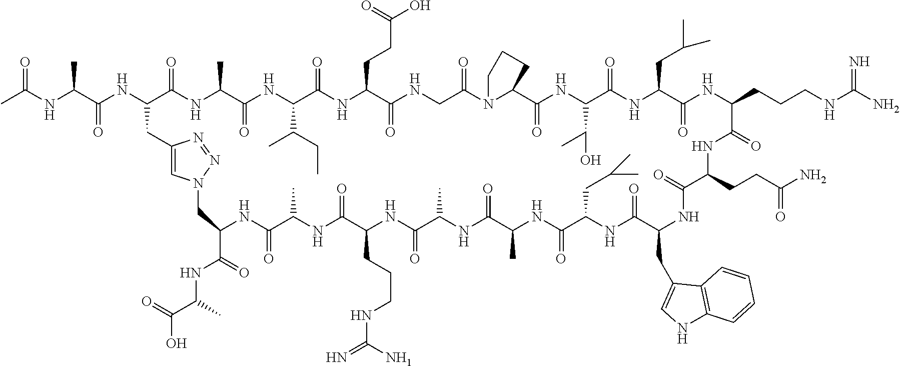

22. The method according to claim 1 wherein the peptidyl TPOR antagonist is:

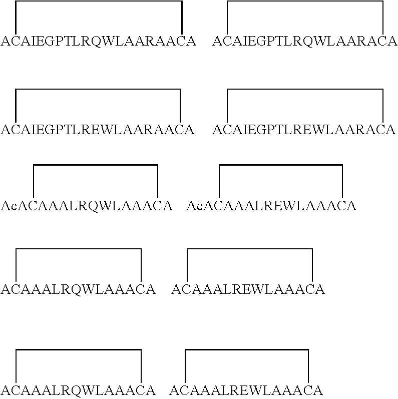

23. The method according to claim 1 wherein the disease or condition associated with signalling via the TPO receptor is a haematological disorder, or other haematological malignancy.

24. (canceled)
